(19) United States
(12) Patent Application Publication
(10) Pub. No.: US 2022/0031955 A1
(43) Pub. Date: Feb. 3, 2022

(54) SYSTEM FOR ENHANCING DATA QUALITY OF DISPENSE DATA SETS

(71) Applicant: Novo Nordisk A/S, Bagsvaerd (DK)

(72) Inventors: Mark Haun, Brier, WA (US); Carsten Schau Andersen, Odense M (DK); Thomas Dedenroth Miller, Copenhagen SV (DK); Ryan Scott Frazier, Lynnwood, WA (US)

(21) Appl. No.: 17/275,722

(22) PCT Filed: Sep. 24, 2019
(86) PCT No.: PCT/EP2019/075626

§ 371 Date: Mar. 12, 2021

Related U.S. Application Data
(60) Provisional application No. 62/735,354, filed on Sep. 24, 2018.

(30) Foreign Application Priority Data
Oct. 3, 2018 (EP) 18198410.5

(51) Int. Cl. A61M 5/315; G16H 40/67; G16H 20/17

(52) U.S. Cl. CPC A61M 5/31535; A61M 5/31565; A61M 2005/3125; G16H 20/17; G16H 40/67

(57) ABSTRACT

A method for enhancing data quality of a drug dose dispense data set to automatically provide dispense data reflecting actually injected dose amounts. For a given dispense session the method comprises the steps of creating a list of possible dispense patterns in accordance with a set of pattern rules and then for each pattern in the list calculate a weight allowing a winning pattern to be determined. Each dispense pattern is a particular sequence of priming events and injection events, with each dispense pattern being a possible interpretation of the dispense events in the current session. For each pattern a combined pattern weight being the product of weight factors for each dispense in the pattern is calculated, wherein each weight factor is determined in accordance with a weight factor vs dispense size function for the given dispense type and dispense size, wherein the larger the dispense size, the more likely it is to represent an injection event and the less likely it is to represent a priming event.

The present invention relates to a system and method for enhancing data quality of a drug dose dispense data set, in order to provide reliable automatic dispense data reflecting actually injected dose amounts.

### BACKGROUND OF THE INVENTION

Decision support systems (DSS) have been proposed, see e.g. PCT/EP2019/067000, designed to help patients titrating up to optimum insulin dose. A fundamental requirement for obtaining a reliable DSS is good data quality in terms of historic injected insulin amounts. Drug delivery devices and add-on devices therefor have been provided adapted to automatically create a log of expelled dose amounts, however, not all captured dispenses are clinically relevant for which reason it is desirable to provide a data filtering solution for pen based basal and bolus therapy that enables filtering of injected insulin doses from non-injected insulin doses.

This issue has also been addressed in the past. For example, WO 2016/007935 discloses an intelligent medicine administration system comprising an injection device, in communication with a patient's smartphone, in which the injection device is able to detect and record dose sizes that are dispensed (e.g., primed or injected to the patient), and to distinguish between a prime dose and a therapy dose. Patients may need to dispense a prime or priming dose prior to injecting the therapy or therapeutic dose. For example, in some use cases, the patient will replace their needle and deliver a prime dose intended to clear the new needle of air. In some cases, for example, a prime dose will be delivered even though the needle was not replaced. In some cases, for example, a prime dose will not be delivered even though the needle was replaced. It is necessary to be able to determine which doses are the prime doses and which are therapeutic doses, in which data associated with the determination of the dose type should be included in the dose calculation (e.g., “Insulin on Board” calculation) and the therapy analytics.

Typically, when a prime dose is delivered, it is followed by a therapy dose. In some implementations of the intelligent medicine administering system, for example, the software application of the smart phone can include a dose distinguisher or identification module to process dose dispensing data and determine and distinguish between a prime dose and a therapy dose that was dispensed from the pen device.

In some implementations of the intelligent medicine administering system, for example, the data processing unit on the pen device can include the dose distinguisher module to process dose dispensing data and determine and distinguish between a prime dose and a therapy dose that was dispensed from the pen device.

In some embodiments, the dose distinguisher module is configured to implement a dose classification method to group data associated with dispensed medicine doses and classify the dispensed doses in the group as either a prime dose or an injected (e.g., therapy) dose, such that, for any group of doses happening in close temporal proximity, only the last dose is recorded as a therapeutic dose. The close temporal proximity is a predetermined temporal threshold value, e.g., which can be defined as 10 seconds, 30 seconds, 1 minute, 2 minutes, 5 minutes, or 10 minutes or other.

Identifying doses as prime is important in patients with low insulin requirements. For example, in a child, a typical prime dose may be 2 units while a typical therapy dose may be 0.5 or 1 units. In this case, if a user were to include all dispensed insulin in the tracking (rather than only the therapeutic insulin), then the therapy tracking would be wrong as would any insulin on board calculations or future dose recommendations.

This is one example showing that prime doses cannot be distinguished based solely on size. In the example given above, the therapy dose is much smaller than the prime dose, but with a fully grown adult or a type 2 patient, the therapy dose may be much larger than the prime.

In some cases, for example, a user may prime their device and not deliver a therapeutic dose. To prevent the dose distinguisher module from improperly identifying the dose as a therapeutic dose, in such cases, the system can include an additional mechanism that may be utilized to quickly identify the dose as either “prime” or “therapeutic”. In one example of this additional dose identification mechanism, a user verification input can be included in the software application of the smart phone to allow the patient to identify that the recorded doses were one of the prime or therapy doses, which would then allow for such doses to be included in any therapy analytics and insulin on board calculation, as appropriate. This user verification input mechanism can include a radio button, a toggle switch, and/or graphic of the user interface allowing tapping on the dose, slider, or other mechanism.

In some embodiments, for example, the dose distinguisher module can be configured to include one or more additional processes or exceptions to the exemplary dose classification method to group and classify the last dose of a group of doses happening in close temporal proximity as a therapeutic dose. In an example, the dose classification method can be implemented such that following a cartridge replacement, if there is only a single dose, it would be designated a prime dose and not a therapy dose. In another example, the dose classification method can be implemented such that when a first dose (or intermediate dose) is larger than a predetermined dose quantity threshold, that dose is considered therapy. For example, any dose determined to be larger than 2, 5, 10 units or other size could be considered therapy regardless of their position in the dose sequence.

The dose distinguisher module of the disclosed systems to determine prime doses from therapeutic doses can include a separate dosing knob on the pen device for prime dosing. The exemplary separate dosing knob can be structured to actuate the dose jackscrew, but not the dose encoder. In these embodiments, for example, when the user rotates the separate dose knob, the medicine is injected but the encoder does not count the dose.

The dose distinguisher module of the disclosed technology to determine prime doses from therapeutic doses may include additional or alternative methods for dose distinguishing. In one example, a method to determine if a dispensed dose is prime or not includes sensing if the pen device is in contact with the body at the time of injection. This can be done in any of several ways. In one nonlimiting example, the pen device 10 can include a pressure sensor coupled to the needle assembly or tip or end of the body of the pen device 10 to determine if a force has been applied at the needle assembly or tip of the device as when injecting. In one non-limiting example, the pen device can include a capacitive sensor fitted near the end of the device which would sense proximity to the body. In either of these exemplary cases, sensing pressure or proximity would result in the dose being considered therapeutic and not prime.

The dose classification method to determine prime doses versus therapy doses can include detecting the speed of doses being delivered. For example, it is possible that prime doses are delivered at a faster rate than non-prime doses. The encoder mechanism of the pen device can be configured to record the speed of the dose, e.g., in which the speed data is transferred to the smart phone for processing. The speed may then be compared to a predetermined dose rate threshold to determine if the dose is a prime or not. For example, the encoder mechanism can detect the speed, where the threshold will depend on the gear ratio, and the encoder counts per revolution and/or other factors. It may be determined that doses resulting in average dose speeds over a pulse per second threshold are prime doses. This dose rate threshold could be determined by asking users to deliver a series of both prime and therapy doses and comparing the average dose speed of each. If there is little overlap in the dose speed ranges of each type of dose then dose speed is a good indicator of type of dose. In some implementations, for example, the dose distinguisher module can utilize the detected dose speed in addition to the dose dispensing groupings within the predetermined amount of time proximity to identify the therapy dose from a prime dose. In some implementations, for example, the dose distinguisher module can utilize the detected dose speed without consideration of the sequence of doses in a dose dispensing grouping.

In some implementations, the dose classification method to determine prime doses versus therapy doses can involve the pen device including a shroud assembly around all or a part of the needle of the needle assembly and a sensor in the shroud assembly. In implementations, when the needle is injected into a patient, the shroud would contact the skin and slide back, triggering the sensor to detect and indicate an actual therapy dose. If the shroud does not move back, it would indicate the pen was being held in the air and the dose would be considered a prime. Alternatively, instead of the shroud, the sensor can be structured in an assembly including a small button or lever that contacts the skin and functions similarly.

In some implementations, the dose classification method to determine prime doses versus therapy doses can involve the pen device including an internal accelerometer, gyroscope, or other rate sensor to detect movement data of the pen device, which is transferred to the smart phone to analyse the movement data. For example, if the pen senses an inward motion before the dose is dispensed and an outward motion after the dose is dispensed, the smart phone would indicate that the pen had been injected into a patient and thereby identify the dispensed dose as a therapy dose, whereas if these motions were absent, it would indicate that the pen had been held in the air.

In some embodiments of the dose distinguishing module, for example, the module can include a ‘voting’ method to determine if a dose is a prime dose. In an illustrative example of the voting method, the dose distinguishing module can implement multiple embodiments of the dose classification method in parallel for a particular dosing sequence, e.g., such as the exemplary dose grouping process (e.g., identifying the last dispensed dose in a sequence of doses dispensed in a predetermined time proximity as the therapy dose), the exemplary dose speed detection process, the exemplary movement data detection process, etc. If after a particular dosing or dosing sequence, a certain majority of the exemplary methods for dose distinguishing indicated that the dispensed dose is a prime dose, and a minority method indicated it is not, then the voting method would determine that in this case the dose would be identified as a prime dose.

Having regard to the above it is an object of the present invention to provide systems, methods and devices adapted to improve data quality of a drug dose dispense data set, in order to reliably and cost-effectively provide automatic dispense data reflecting actually injected dose amounts.

The improved data could be used in a DSS in order to better, faster and more precisely provide dose guidance for a patient, e.g. in a system adapted to provide insulin adjustment day dose recommendation (titration) for a subject to treat diabetes mellitus. Alternatively, the system in accordance with the invention could assist a patient in keeping an electronic dose log (e.g. provided by a dose logging app running on a smartphone and receiving data from a connected drug delivery device) in which dose events are automatically labelled an injected or non-injected amounts, this removing the burden and complexity of this task from the patient as well as providing reliable dose data to help health care persons and patients to work together to provide better adherence. For such applications the DSS and the dose logging app can be considered clients to the service provided by the current invention.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention a computing system for enhancing data quality of a query dispense data set is provided. The system comprises one or more processors and a memory in which instructions are stored that, when executed by the one or more processors, perform a method responsive to receiving a query request for enhancing dispense data quality from a client. The method comprising the steps of (a) obtaining a query dispense data set comprising a plurality of dispense records created over a time course, each respective dispense record representing a dispense event comprising (i) a dispense amount of a medicament, wherein the dispense amount is one of a priming amount [p] or an injection amount [i], each amount corresponding to a size, (ii) a corresponding dispense timestamp, and the further step of (b) segmenting the query dispense data set into one or more current sessions, each current session comprising a sequence of dispense events clustered in time according to a set of clustering criterions. For each current session the method comprises the steps of (c) creating a list of possible dispense patterns in accordance with a set of pattern rules, wherein a dispense pattern is a sequence of dispense amounts, either priming or injection amounts, (d) for each pattern calculating a combined pattern weight being the product of weight factors for each dispense in the pattern, wherein each weight factor is determined in accordance with a weight factor vs dispense size function for the given dispense type and dispense size, wherein the larger the dispense size, the more likely it is to represent an injection event and the less likely it is to represent a priming event, (e) identifying a winning pattern as the pattern having the highest combined pattern weight, and (f) storing in memory the corresponding dispense events labelled as either a priming or an injection event corresponding to the winning pattern.

When it is defined that the query dispense data set is segmented into one or more current sessions, this includes that the dispense data set allows no session to be identified. Further, segmentation of the query dispense data set into sessions includes cases in which a data set has been obtained in an already segmented format which may then be accepted or segmented again using the rules of the claimed method.

As appears, the method is based on the concept of creating a list of possible dispense patterns in accordance with a set of pattern rules and then for each pattern in the list calculate a weight allowing a winning pattern to be determined. More specifically, each dispense pattern is a particular sequence of priming events and injection events, with each dispense pattern being a possible interpretation of the dispense events in the current session. The calculation is based on the realization that the larger the dispense size, the more likely it is to represent an injection event and the less likely it is to represent a priming event. Based on this weight factor vs dispense size functions can be created for each priming amount [p] and each injection amount [i] in each pattern.

As is clear to the skilled person, setting out the actual rules and determining the actual weight factor vs dispense size functions can be done in numerous ways and in accordance with normal design procedures for a data handling system as well as the design guidance provided in this application.

By the above steps a method is provided allowing dispense events to be labeled with high reliability and flexibility. An estimated total injected amount for a session can be calculated as the sum of all injection amounts in the winning pattern.

This said, in exemplary embodiments a given event or session label can be changed by a user, which label is then accepted by the system for future calculations.

In order to further refine reliability and precision of the method a history dispense data set comprising a plurality of prior dispense records created over a prior time course may be obtained, e.g. from data stored in previous query requests.

The combined pattern weight may be the product of one or more additional factors from the group comprising: priming probability factor based on history dispense data, priming disparity factor (for bolus injections), and intra-session dispense interval factor for sessions having more than two dispenses. When attempting to distinguish primes from the small injections which are common in bolusdrug patients, it can be helpful to down-weight patterns where the priming dispenses do not have a consistent size by applying a priming disparity factor, this being based on the realizations that priming doses are more likely to be of constant size.

In an exemplary embodiment the method comprises the further steps for each current session: generating mean and variance values for an expected total injected amount distribution based on history dispense data, and comparing the highest and the second-highest combined pattern weights, and if the pattern weights are within a given proximity of each other, then identify an updated winning pattern as the pattern having the highest probability according to the generated distribution. Indeed, if there is more than one close candidate to have the second-highest combined pattern weights, then also these weights should be compared.

By incorporating the use of historic data and calculate mean and variance values for an expected total injected amount distribution, the method is able to more reliably identify a winning pattern in cases where two (or more) pattern weights are within a given proximity of each other.

To further improve reliability and precision the method may comprise the further step for each current session: calculating a history weight for the history dispense data upon which the expected total injected amount values are based. The history weight may be based on relevance criteria comprising one or more of age of data, time-of-day similarity, and inter-session gap similarity.

In other words, the more similar prior data values are to current data values the more weight they provide to the calculation. Thus, unless the history weight reaches a given minimum threshold no expected total injected amount values are generated.

To further improve reliability and precision the method may comprise the further step for each current session: determining a combined confidence value based on one or more confidence metrics from the group of confidence values comprising: data confidence value based on the value of the highest combined pattern weight (i.e. the higher the value the higher the confidence), expected-amount confidence value based on the difference between estimated total injected amount and an expected total injected amount, if calculated, ambiguity confidence value based on the probability proximity of the highest and the second-highest combined pattern weights according to the generated distribution, if generated (i.e. when two patterns are almost equally likely, then ambiguity confidence is low), and priming confidence value based on the consistency between priming behavior of the winning pattern (i.e. the more the user has primed in the past, the higher the confidence of an assumed priming event). The combined confidence value can be calculated in different ways, e.g. as a mean of all values or as the single lowest value. When the combined confidence value is above a given threshold value, then an estimated total injected amount can be calculated as the sum of all injection amounts in the winning pattern. The estimated total injected amount can then be provided to the requester, e.g. the patients personal log or a DSS for use in further calculations.

Alternatively, the estimated total injected amount (irrespective of the size of the combined confidence value) may be provided in combination with the combined confidence value, this allowing a patient or system to evaluate the result in view of confidence level.

Further, when the combined confidence value is above a given threshold value for a current session, the session can be labeled as such, whereby the mean and variance values for the expected total injected amount distribution is based on history dispense data from labeled sessions only, this providing improved reliability and precision of the calculated values. When a session is not labelled, the system may prompt the user to label the session allowing the session to be used for future calculations.

The combined pattern weight may be the product of one or more further factors from the group comprising: priming probability factor based on history dispense data, priming disparity factor, and intra-session dispense interval factor for sessions having more than two dispenses

In exemplary embodiments the obtained dispense records comprise an identifier for identifying a given dispense event as a bolus event or a basal event, this allowing the rules and parameters of the method to be adapted for use with dispense data generated in a bolus only, basal only, or bolus and basal regimen.

The segmenting may be controlled by a set of time parameters and a set of time measures, wherein the initial dispense event in the sequence of dispense events starts a session and zeros a timer, and the next dispenses are automatically included in this session until a session time window have elapsed, and wherein later dispenses are included, provided that the expressions: (i) the ratio between a resulting session length and the resulting inter-session length on either side of the session is less than the session length ratio, and (ii) the resulting session length is less than session window max, is true, wherein the sequence of dispense events in the session defines a set of dispense events, and wherein each dispense event comprises a corresponding dispense size being the amount of dispensed medicament, and wherein a new session is started, in response to the expressions are no longer true.

In a specific aspect of the invention a computing system for enhancing data quality of a query dispense data set is provided. The system comprises one or more processors and a memory in which instructions are stored that, when executed by the one or more processors, perform a method responsive to receiving a client request for enhancing dispense data quality of a dispense data, the method comprising the steps of:
obtaining a dispense data set from one or more injection devices used by the subject to apply the treatment regimen, the dispense data set comprising a plurality of dispense records taken over a time course, each respective dispense record in the plurality of dispense records comprises: (i) a respective dispense event including an automatically obtained amount of medicament dispensed by the subject using a respective injection device in the one or more injection devices, wherein the dispense event is one of a priming event of an injection event, wherein a priming event is any dispensing event preparatory to an injection event, and an injection event is a dispense event, wherein the medicament is presumed injected into the subject, (ii) a corresponding automatically obtained dispense event timestamp within the time course that is automatically generated by the respective injection device upon occurrence of the respective medicament dispense event,
segmenting the dispense data set into a plurality of segments, wherein each segment comprises a session,
wherein each respective session comprises a sequence of dispense events of the dispense data set clustered in time, during which time the user intends to perform one or more injections events and wherein one or more of the dispense events in the sequence of dispense events can be interpreted as an injection event,

for a current session:
obtaining (i) an expected dose and a corresponding variance based on prior sessions, and by setting time weights wherein the time weights is related to similarity in time of day, inter-session time and session age, or
obtaining (ii) a guided dose and a corresponding variance based on dose guidance, and thereby:
providing a prior dose based probability distribution for a session-dose,
listing a set of allowed dispense patterns, wherein a dispense pattern is a particular sequence of priming events and injection events, and whereby each dispense pattern is an interpretation of the dispense events in the current session, which together with the amount of dispensed medicament can provide an estimate of the session injected dose,
calculating pattern weights based on a priming probability of prior sessions or intra-session time length between dispense events in the set of dispense events of the current session,
calculating pattern probabilities based on the dispense size, wherein the larger the dispense size, the more likely it is to be an injection event and the less likely it is to be a priming event,
calculating a combined pattern probability based on the pattern weights and the pattern probability based on dispense size,
listing the possible doses based on the dispense size, wherein a possible dose is a combination of one or more of the dispense events, which are assumed to be an injection event,
for each of the possible doses, identifying the one or more possible patterns and the corresponding combined pattern probability, and calculating the sum of the one or more corresponding combined pattern probabilities to provide a sum of combined pattern probability,
for each of the possible doses, calculating the combined probability of possible doses based on the sum of combined pattern probability and a corresponding dose prior obtained from the prior dose based probability distribution,
wherein the possible dose resulting in the largest combined probability of possible doses is the most likely session-dose designated the Maximum Likelihood dose, wherein the provision of the most likely session-dose of each session enhances the quality of the dispense data set and enable reliable automatic decision support.

In this way technical information comprising pattern probabilities based on the dispense size can be used to automatically enhance the quality of the dispense data set. The technical information on pattern probability can be combined with different pattern weights, and is thereafter converted to a session-dose probability which can be combined with a prior probability distribution of the session-dose. The enhanced data quality of the dispense data set, comprising a Maximum Likelihood estimate of the dose session, can be used as input in further steps of the decision support system, wherein a data set with an enhanced quality provides an enhanced quality of the final data output of the decision support system, compared to the output of based on non-enhanced dispense data.

The segmenting may be controlled by a set of time parameters and a set of time measures, wherein the initial dispense event in the sequence of dispense events starts a session and zeros a timer, and the next dispenses are automatically included in this session until the session time window have elapsed, and wherein later dispenses are included, provided that the expressions: (i) the ratio between a resulting session length and the resulting inter-session length on either side of the session is less than the session length ratio, and (ii) the resulting session length is less than session window max, is true,
wherein the sequence of dispense events in the session defines a set of dispense events, and wherein each dispense event comprises a corresponding dispense size being the amount of dispensed medicament, and
wherein a new session is started, in response to the expressions are no longer true.

In a further aspect the method further comprises: calculating a set of confidence scores for the Maximum Likelihood dose, evaluating whether the smallest confidence score is larger than a confidence threshold, and automatically labeling the session in response to the confidence evaluation is true.

In a further aspect the labeling comprises assigning a dispense pattern to the current session, wherein the assigned dispense pattern is the most likely dispense pattern of the one or more dispense patterns resulting in the Maximum Likelihood dose.

In a further aspect the method further comprises: requesting the user to user to confirm a labeling step on unlabeled sessions, in response to the confidence evaluation being false.

In a further aspect, the method further comprises: automatically providing a recommended dose based on one or more of the estimated Maximum Likely Hood doses.

### BRIEF DESCRIPTION OF THE DRAWINGS

In the following embodiments of the invention will be described with reference to the drawings, wherein

FIG. 1A shows a pen device,

FIG. 1B shows the pen device of FIG. 1A with the pen cap removed,

FIGS. 1C and 1D show a schematic representation of an add-on device to collect dose dispense data from a drug delivery device, the drug delivery device also being shown on the figures,

FIGS. 2A-2G collectively illustrate an exemplification of a method of enhancing dispense data quality based on basal sessions from a basal study data set for a patient,

FIGS. 3A-3D collectively illustrate an exemplification of a method of enhancing dispense data quality based on bolus sessions from a bolus study data set for a patient,

FIG. 4A shows a flow-chart for an exemplary algorithm,

FIG. 4B show potential patterns for different dispenses per session,

FIG. 4C shows an example of a weight factor vs dispense size function,

FIG. 4D shows potential dose size outcomes for different sessions patterns,

FIG. 5A illustrates an exemplary system topology that includes a decision support system for processing the stream of collected data from a data collection device; as shown the data collection device can collect data from one or more injection devices, and in some embodiments, it can also collect blood glucose data from one or more glucose sensors that measure glucose data from the subject, the one or more injection devices are used by the subject to inject blood glucose regulating medicaments in accordance with a treatment regimen, where the above-identified components are interconnected, optionally through a communications network, in accordance with an embodiment of the present disclosure,

FIG. 5B illustrates a decision support system in accordance with an embodiment of the present disclosure, the decision support system comprises a processor and a memory, wherein the system is adapted for enhancing the data quality of dispense data obtained from one or more injection devices,

FIG. 5C illustrates a method according to the present disclosure for enhancing data quality of dispense data obtained from a data collection device, wherein the data with the enhanced data quality is structured for use by a decision support system,

FIGS. 6-17 collectively illustrates in general aspects the method of enhancing dispense data quality, which is illustrated in FIG. 5C,

FIGS. 6 and 7 collectively illustrate a step of segmenting data in the method of enhancing dispense data quality, which is illustrated in FIG. 5C,

FIGS. 8A, 8B, 8C and 8D collectively illustrate a step of determining an expected dose based on information of prior sessions,

FIGS. 9A and 9B collectively illustrate a step of obtaining dose probabilities based on time data from prior sessions,

FIG. 10 illustrates a step of obtaining allowable dispense data based on number of dispenses and selection rules,

FIGS. 11A and 11B collectively a step of setting pattern weights for the allowable patterns,

FIGS. 12A and 12B collectively illustrate a step of calculating pattern probabilities of the allowable patterns based on dispense size,

FIG. 13 illustrates a further step of updating pattern probabilities,

FIG. 14 illustrates a further step of converting pattern probabilities into dose probabilities,

FIG. 15 illustrates a further step of updating dose probabilities with dose probabilities based on time data from prior sessions,

FIG. 16 illustrates a further step of calculating confidence scores based on confidence metrics,

FIG. 17 illustrates a further step of deciding whether or not to label the session,

FIG. 18 illustrates a patient survey plot comprising a number of dispense sessions, and

FIGS. 19A-19K collectively illustrate an exemplification of the method of enhancing dispense data quality based on sessions from the FIG. 18 plot.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member is used for a given component it can be used to define a unitary component or a portion of a component, having one or more functions.

In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

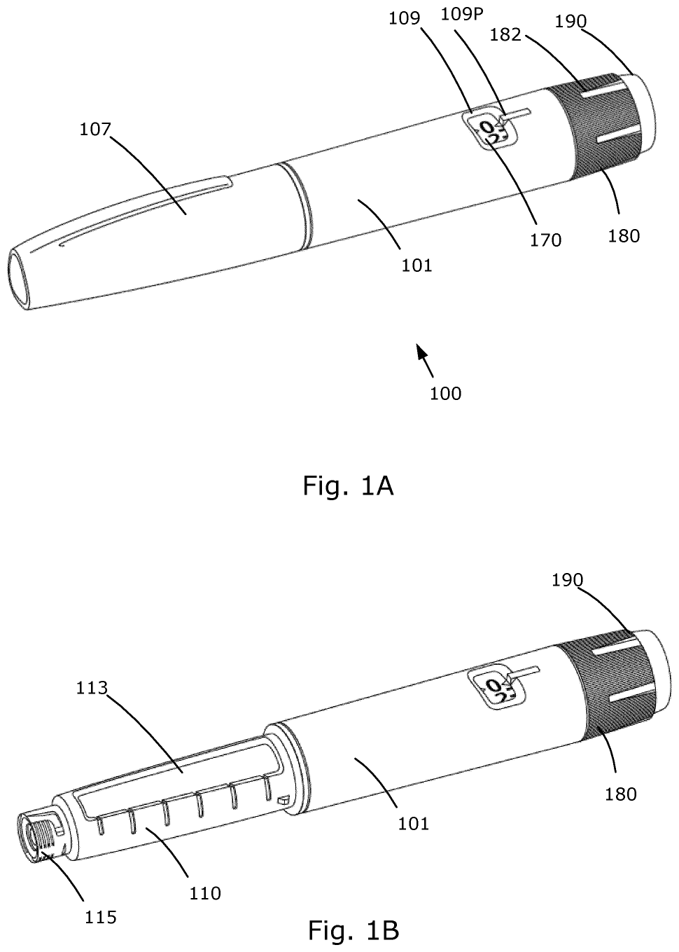

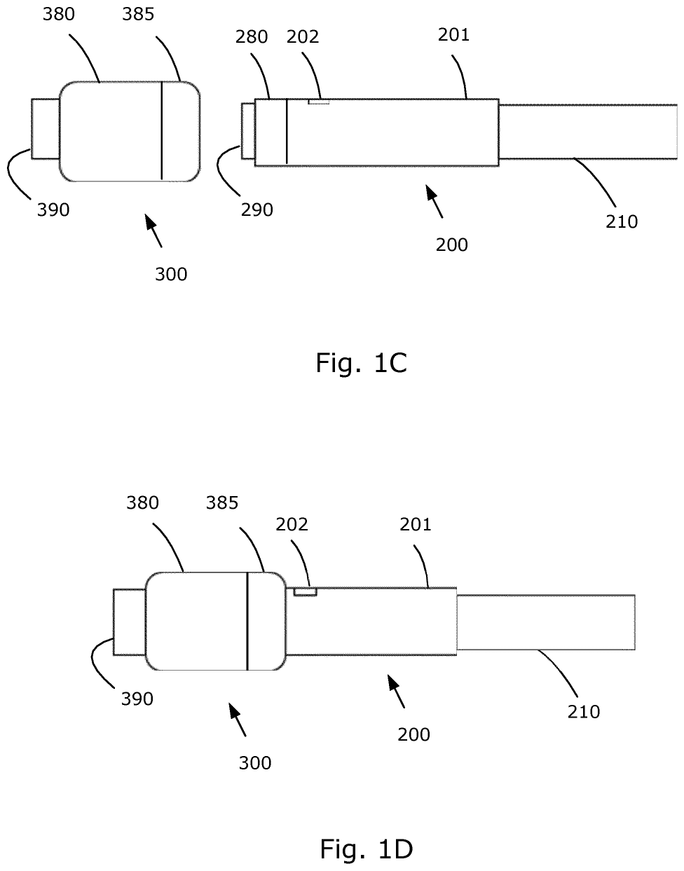

Before turning to embodiments of the present invention per se, an example of a prefilled drug delivery device will be described, such a device providing the basis for the exemplary embodiments of the present invention. Although the pen-formed drug delivery device 100 shown in FIGS. 1-3 may represent a “generic” drug delivery device, the actually shown device is a FlexTouch® prefilled drug delivery pen as manufactured and sold by Novo Nordisk A/S, Bagsværd, Denmark.

The pen device 100 comprises a cap part 107 and a main part having a proximal body or drive assembly portion with a housing 101 in which a drug expelling mechanism is arranged or integrated, and a distal cartridge holder portion in which a drug-filled transparent cartridge 113 with a distal needle-penetrable septum is arranged and retained in place by a non-removable cartridge holder attached to the proximal portion, the cartridge holder having openings allowing a portion of the cartridge to be inspected as well as distal coupling means 115 allowing a needle assembly to be releasably mounted. The cartridge is provided with a piston driven by a piston rod forming part of the expelling mechanism and may for example contain an insulin, GLP-1 or growth hormone formulation. A proximal-most rotatable dose setting member 180 with a number of axially oriented grooves 182 serves to manually set a desired dose of drug shown in display window 102 and which can then be expelled when the button 190 is actuated. The window is in the form of an opening in the housing surrounded by a chamfered edge portion 109 and a dose pointer 109P, the window allowing a portion of a helically rotatable indicator member 170 (scale drum) to be observed. Depending on the type of expelling mechanism embodied in the drug delivery device, the expelling mechanism may comprise a spring as in the shown embodiment which is strained during dose setting and then released to drive the piston rod when the release button is actuated. Alternatively the expelling mechanism may be fully manual in which case the dose member and the actuation button move proximally during dose setting corresponding to the set dose size, and then is moved distally by the user to expel the set dose, e.g. as in a FlexPen® manufactured and sold by Novo Nordisk A/S.

Although FIGS. 1A and 1B show a drug delivery device of the prefilled type, i.e. it is supplied with a pre-mounted cartridge and is to be discarded when the cartridge has been emptied, in alternative embodiments the drug delivery device may be designed to allow a loaded cartridge to be replaced, e.g. in the form of a “rear-loaded” drug delivery device in which the cartridge holder is adapted to be removed from the device main portion, or alternatively in the form of a “front-loaded” device in which a cartridge is inserted through a distal opening in the cartridge holder which is non-removable attached to the main part of the device.

FIGS. 1C and 1D show a schematic representation of an assembly of a pre-filled pen-formed drug delivery device 200 and a therefor adapted add-on dose logging device 300. The add-on device is adapted to be mounted on the proximal end portion of the pen device housing and is provided with dose setting and dose release means 380 covering the corresponding means on the pen device in a mounted state as shown in FIG. 1D. In the shown embodiment the add-on device comprises a coupling portion 385 adapted to be mounted axially and rotationally locked on the drug delivery housing. The add-on device comprises a rotatable dose setting member 380 which during dose setting is directly or indirectly coupled to the pen dose setting member 280 such that rotational movement of the add-on dose setting member in either direction is transferred to the pen dose setting member. In order to reduce influences from the outside during dose expelling and dose size determination, the outer add-on dose setting member 380 can be rotationally decoupled from the pen dose setting member 280 during dose expelling. The add-on device further comprises a dose release member 390, which can be moved distally to thereby actuate the pen release member 290. The add-on dose setting member 390 gripped and rotated by the user may be attached directly to the pen housing in rotational engagement therewith. WO/2019/162235 discloses an exemplary add-on dose logging device. An example of a drug delivery pen device with integrated dose logging circuitry and wireless communication is the NovoPen® 6 manufactured and sold by Novo Nordisk A/S.

Before a general description of an algorithm providing the initially described functionalities for a data quality enhancing system is given, two walk-through examples covering basal respectively bolus sessions of insulin dispenses will be given.

### Example 1: Basal Walk-Through

The basal walk-through covers seven sessions for user #8511 in an insulin basal study data set (in project “Mustang”). The walk-through ends with session 11 because it shows several important features of the algorithm. The example utilizes prior data as well as confidence values and additional pattern weights.

The example starts with session 5 as sessions 1-4 and the associated calculations are similar to session 5. Further, in the shown example sessions 1-4 are not labeled corresponding to the ignoreFirstSessions parameter, see below.

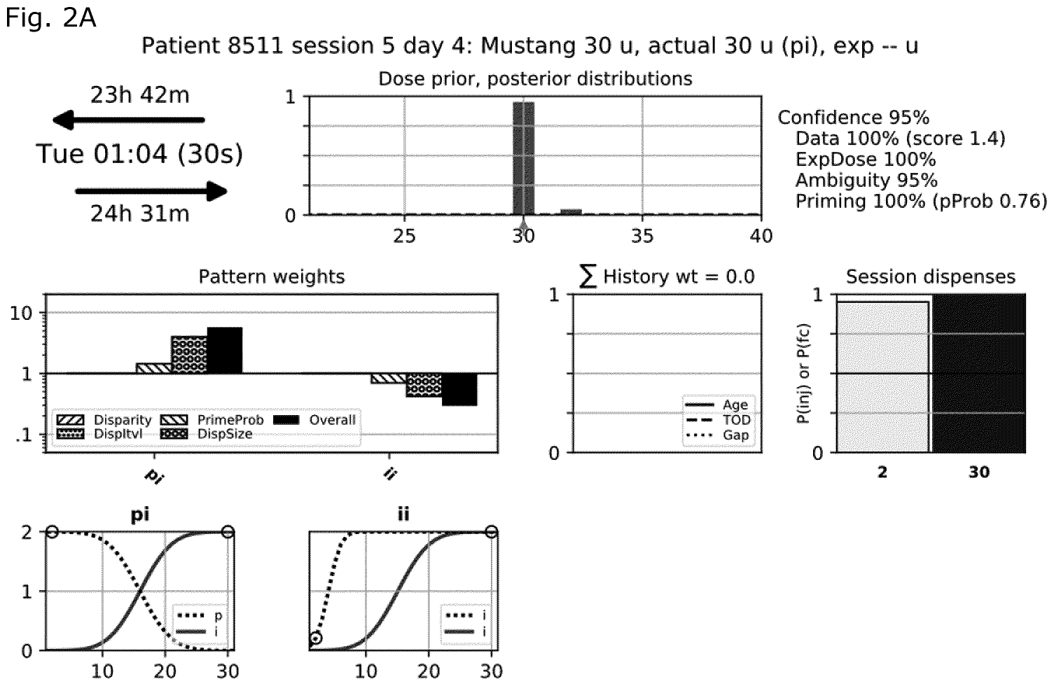

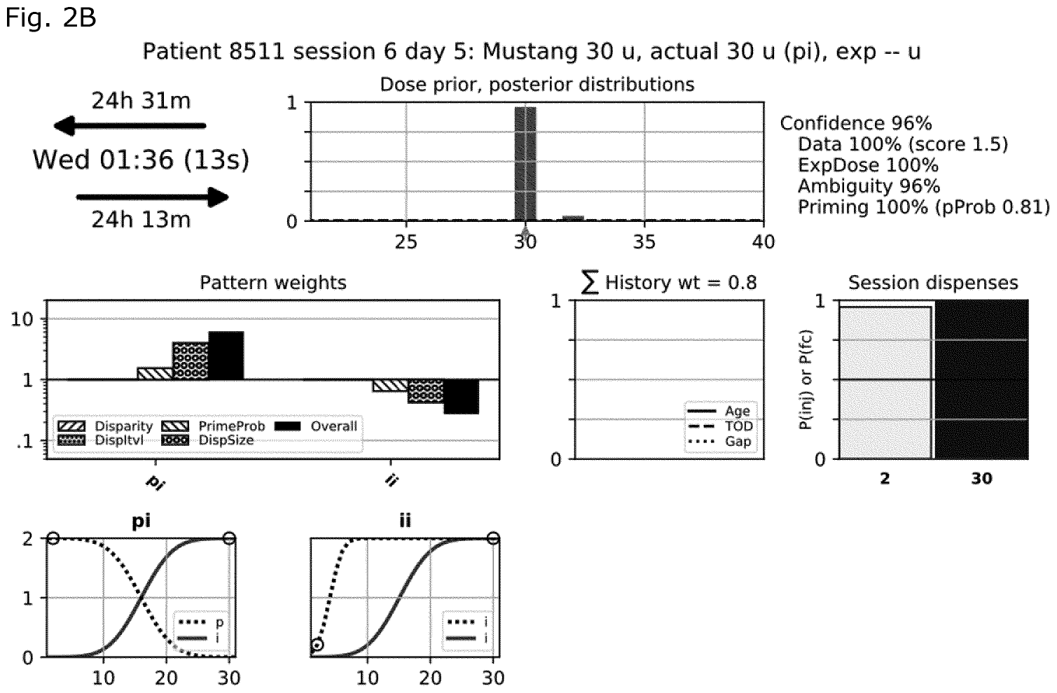

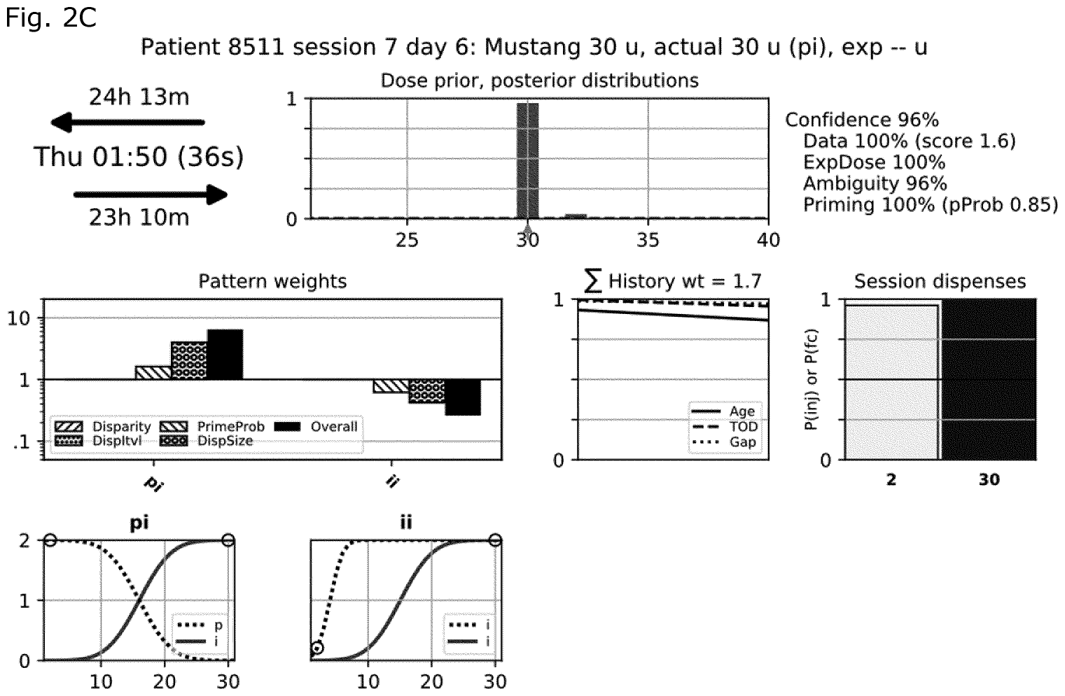

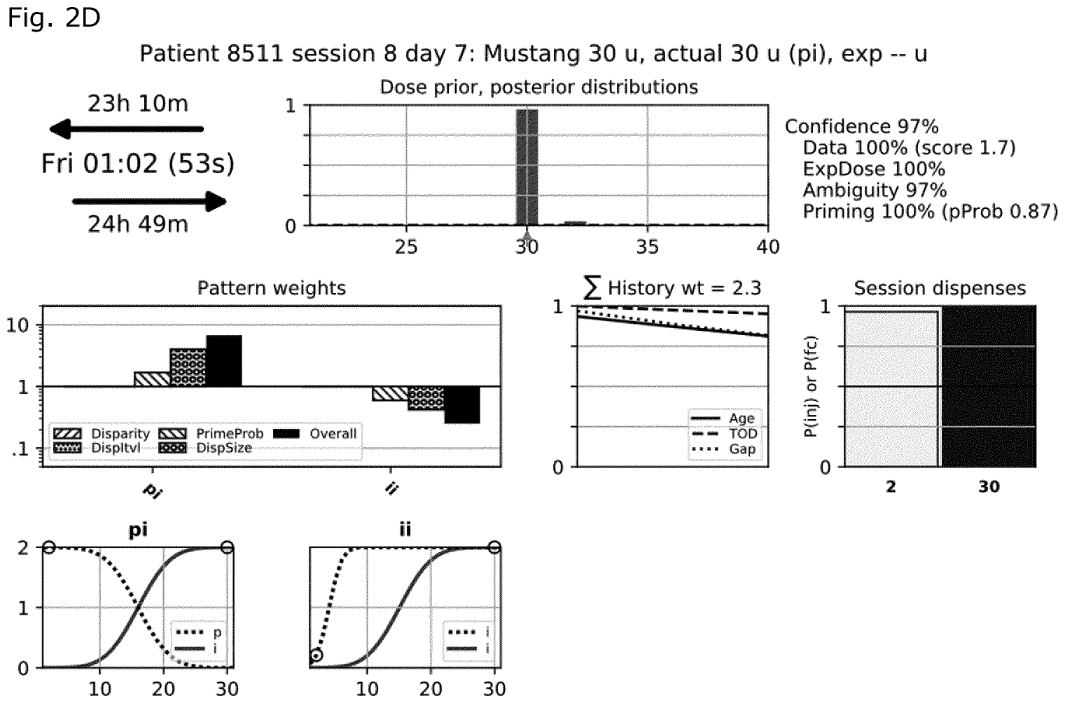

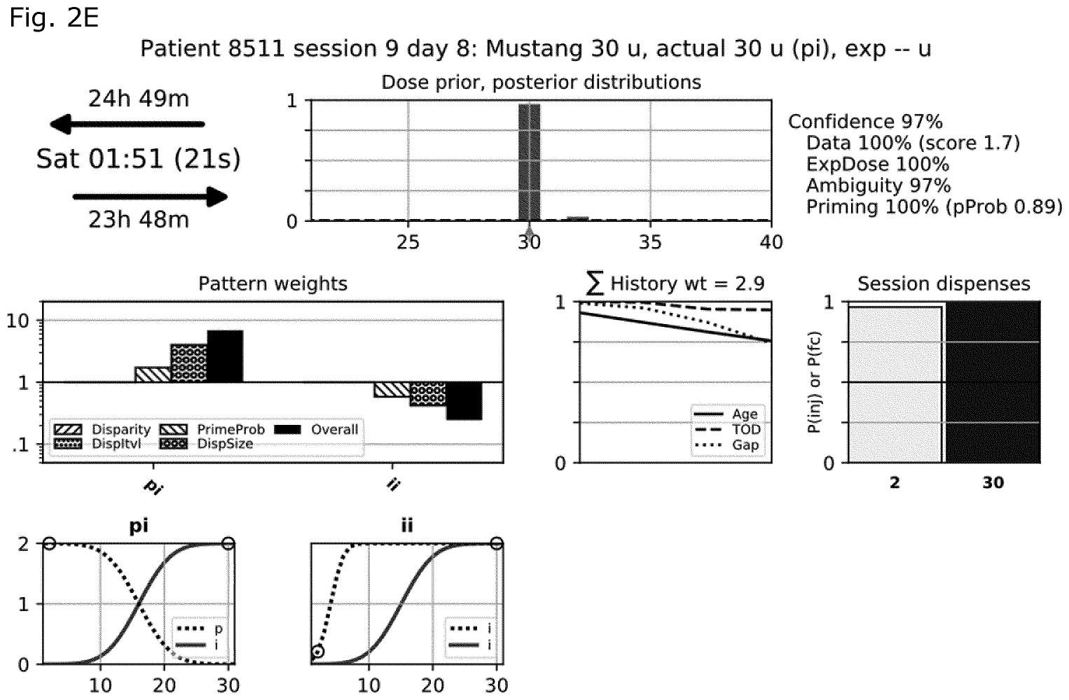

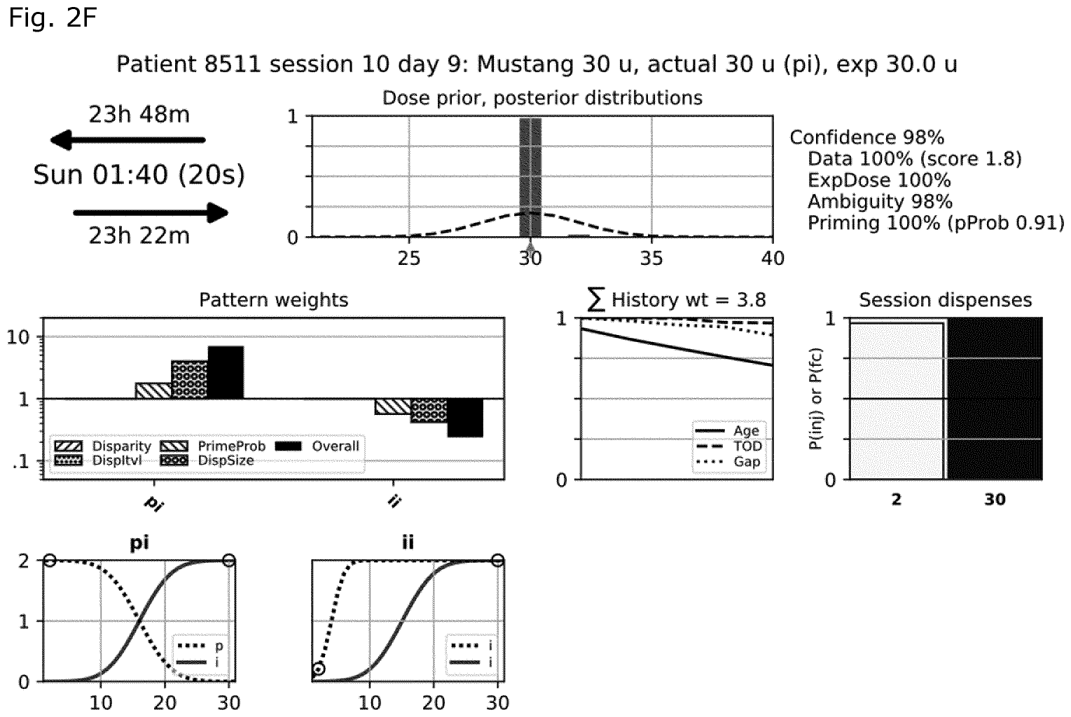

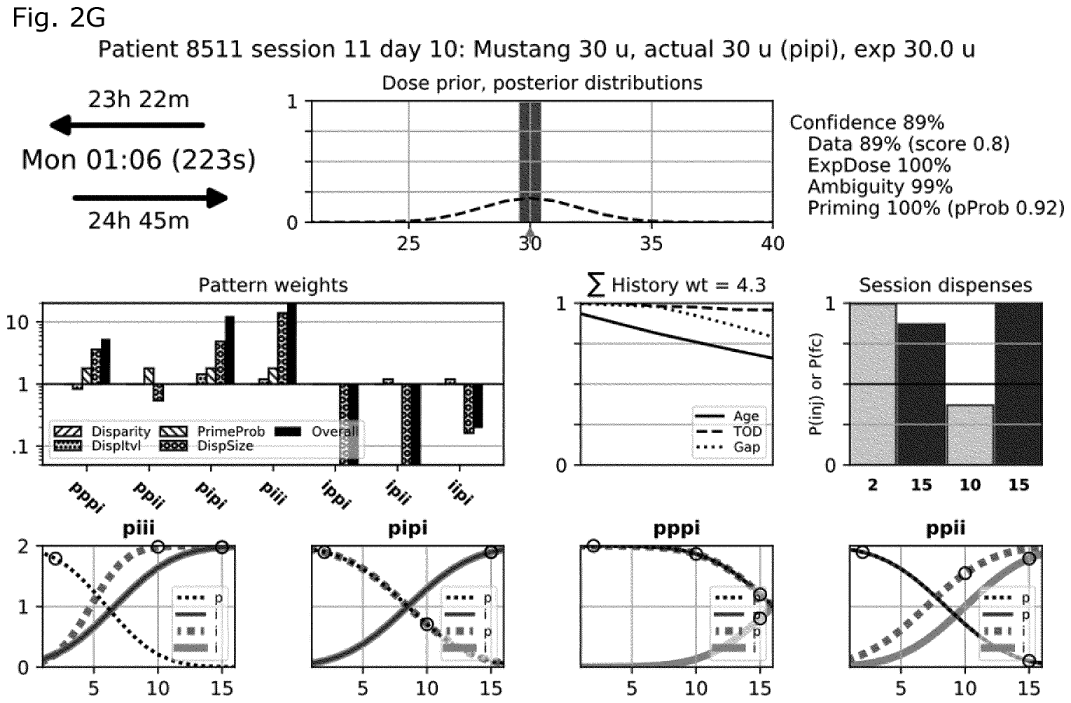

Session 5—FIG. 2A

The session comprises two insulin dose records {2,30}. As indicated in the top left corner the session was recorded a Tuesday at 01:04 (i.e. in the night) and lasted 30 seconds. The time since the previous session was 23 h 42 m and the time to the next session (in the basal study set) was 24 h 31 m. According to rules for “pattern enumeration” there are only two possible interpretations (patterns): pi and ii. These possibilities are evaluated by various criteria summarized in the “pattern weights” subplot:

The pattern pi involves priming whereas ii does not. This user tends to prime (pProb=0.76 in the text block at upper right), so pi gets a priming weight factor >1 and ii gets a priming weight factor <1 (hatched-down bars, PrimeProb).

Looking at dispense sizes, pi is a more likely pattern than ii because the 2-unit dispense is very small for an injection, but just what we would expect for a prime. The size-based analysis is shown in the bottom row of curves, which plot weight factor vs dispense size, the bubbles indicate the sampling points on these curves, at the dispense sizes of 2 and 30. The rules for the generation of these curves, which depend on several configurable parameters, are detailed below in the description of an exemplary algorithm. Notice that in the ii case, the 2-unit “candidate injection”, although it still scores very low, has a curve (dotted) which rises much quicker than the 30-unit candidate injection (solid). This is intentional, to correctly interpret split doses where the two injections are of dissimilar size. The product of the bubbles (about 4 for pi and 0.4 for ii) constitute the size-based weight factors (circle-pattern bars, DispSize).

The Disparity weight factor (hatched-up bars, Disparity) is not used for basal insulin. The intra-session interval analysis (dotted bars, Displtvl) is used for basal insulin but only for sessions of three or more dispenses. Therefore, the overall pattern weights (solid bars, Overall) are simply the product of the size weights and the priming weights.

Notice that there is no expected dose for this session, as evidenced by the empty history subplot and the “exp—u” in the overall plot title. There is no expected dose because there is not enough history to calculate one: The expected dose is a weighted average of the doses from past labeled sessions which meet certain similarity criteria, and the algorithm will not declare an expected dose until the sum of those weights reach some minimum threshold (a configurable parameter, in this case 3.0). Here the sum of history weights is 0.0, because there are no previous labeled sessions.

The top graph with the dark-grey bars shows the final probability (“the posterior distribution”) of dose size for this session. This is just the pattern weights, translated into dose size (pi would be a dose of 30 whereas ii would be a dose of 32), and normalized so that it sums to one. If there were an expected dose for this session, it would manifest as a Gaussian “prior distribution” centered on the expected dose, with variance based on the variance estimate of the expected dose, this would be plotted as a dotted curve on top of the dark-grey bars. If there were a prior, it would be multiplied by the pattern-weight result before the normalization. Because there is no expected dose in this case, the prior is taken as a uniform distribution and thus has no effect on the dark-grey bars, which we call the posterior.

The estimated dose is 30 units. The algorithm will formally label the session because its confidence in the result is greater than the confidence threshold (a configurable parameter, here 70%). The confidence is the minimum of four metrics, as shown in the text block to the right of the dark-grey bar graph. The Data Confidence is measure of the overall plausibility of the winning pattern (here, pi), it is equal to the green bar of the winning pattern, normalized by the number of dispenses in the session. The Expected-Dose Confidence depends on the difference between the expected dose and the estimated dose (not applicable to this session because there was no expected dose). The Ambiguity Confidence depends on the “peakiness” of the peak in the posterior distribution, if the max of the distribution, aka the estimated dose (here 30), has a probability too close to one of the other doses (here 32), the ambiguity is said to be high and the Ambiguity Confidence will suffer. The Priming Confidence measures the consistency between priming behavior in the winning pattern and the priming probability calculated based on past behavior (here 0.76). All four metrics have configurable weights, and in this simulation the Priming Confidence has been turned off, so it will always read 100%.

Session 6—FIG. 2B

The session analysis is nearly identical to session 5. Notice that the priming probability is climbing (now 0.81), which makes the hatched-down PrimeProb bars in the pattern weights slightly higher (for pi) and lower (for ii) than before. This makes the overall pattern weights slightly more distinct, resulting in a slightly larger probability difference between 30 and 32 units in the posterior distribution. (Notice that the Ambiguity Confidence is 96% vs 95% in session 5, and the data score is also higher.) There is still no expected dose, but history is beginning to accumulate (sum of weights=0.8).

Sessions 7-9—FIGS. 2C-2E

These sessions continue the trend from session 6. The user's behavior is exactly the same, so by session 9 the priming probability has risen to 0.89 and the history weights have reached 2.9—almost the 3.0 required to have an expected dose. Confidence is also rising but more slowly, as it approaches a saturation level.

The curves in the history-weight subplot show the similarity criteria used in the (eventual) weighted average of past sessions: Age is a simple exponential decay which weights recent sessions more than older sessions, TOD stands for time of day which preferentially weights past sessions which happened at a similar time of day as the current session, and Gap refers to the time since the last session (the “preceding gap”), which preferentially weights past sessions having a similar preceding gap as the current session's preceding gap.

Session 10—FIG. 2F

User behavior is still the same, and now there is enough history to declare an expected dose: 30 units. There is now a Gaussian prior (dotted curve) centered at 30 units, which will tend to suppress dose possibilities which are far from the expected 30 units. This makes the algorithm more robust when the session dispenses are not so clear-cut as this, as will become evident in the next session. The variance of the Gaussian prior is constrained to be at least “minVariance” (a configurable parameter), this is necessary because even though the user may be 100% consistent at one dose, as here, the user could still titrate to a different dose size at any time, and the algorithm needs to accommodate these gradual changes.

The history weights are as follows (sessions 5-9 inclusive, as all were labeled):

Age: [0.706, 0.758, 0.813, 0.869, 0.934]

Time-of-day: [0.972, 1.000, 0.998, 0.968, 0.997]

Gap from last session: [0.999, 0.944, 0.982, 0.957, 0.892]

Overall (product): [0.685, 0.715, 0.796, 0.805, 0.831] (sum=3.83)

Session 11—FIG. 2G

The dispense pattern {2,15,10,15} is a classic split dose with a twist: Is the third dispense a prime or an injection? The algorithm considers multiple criteria which work in concert to arrive at an answer.

The pattern enumeration results in seven different patterns which need to be considered. (Aspects of the pattern enumeration are configurable parameters.) At a glance, the solid bars in the pattern-weights subplot show that piii has the highest weight, followed by pipi, then pppi. The other possibilities are weighted much lower. Notice that there is only room to show four of the size-based weight curves at the bottom, so the top four patterns are shown.

The top patterns all score high on priming (hatched-down bars, PrimeProb), because the user's priming probability is 0.92 and all of these patterns do involve priming.

The pattern piii gets the highest size-based weight, because the third dispense, when taken as a candidate injection, has a [dotted-grey] curve which is already up at the maximum by 10 units. (Note that the grey curve overlays the solid curve as they are both 15-unit candidate injections.) The pattern pipi has a much lower size-based weight, but notice that the crossover point between the curves for the candidate injections and candidate primes is around eight units. This crossover is adaptive depending on the sizes of the dispenses in the session, and it means that the 10-unit candidate prime at least gets a chance.

For basal, the crossover point for the flow check S-curve (performed via the use of the error function, commonly used in probability and statistics and abbreviated erf( ), see below) is calculated per a specific equation based on the suspected dose, the number of injections in the session, and the historical average flow check size. The suspected dose is based on the expected dose from the history component, the historical average injection size, and the largest dispense value in the current session. All of these factors allow the cross-over point of the S curve to change for that specific user based on their history.

The intra-session interval analysis (dotted bars, Displtvl) comes into play because this session has more than two dispenses. This analysis is based on statistical observations that the time delay when switching from a series of one or more primes to an injection is usually longer than the time delay between primes. Although both pipi and piii have greater-than-one Displtvl weight factors on this basis, pipi has the highest weight factor, which somewhat mitigates its low size-based weight factor. The pattern pppi gets a less-than-one Displtvl weight factor, which further diminishes its overall pattern weight. (Note that pppi and pipi had roughly similar priming and size-based weight factors.)

The history weights are as follows (sessions 5-10 inclusive, as all were labeled):

Age: [0.660, 0.708, 0.759, 0.812, 0.872, 0.935]

Time-of-day: [1.000, 0.981, 0.960, 1.000, 0.957, 0.975]

Gap from last session: [0.987, 0.861, 0.923, 0.996, 0.791, 0.979]

Overall (product): [0.651, 0.598, 0.673, 0.808, 0.661, 0.892] (sum=4.28)

The winning pattern on the basis of pattern weights alone is still piii, which is incorrect, with pipi a close second. If there were no expected dose, the Ambiguity Confidence would be low because of the closeness in probability between 30 (pipi) and 40 (piii) unit doses. Fortunately, there is an expected dose, which reduces the probability of a 40-unit dose to virtually zero and makes 30 (pipi) the winner. Notice that the limiting factor in the confidence score is the Data Confidence at 89%, this is still good, but lower than normal because of the overall mediocre pattern weight of pipi. This is a protective feature to keep the algorithm from being too confident if it gets “saved” by the expected dose, as here, but that pattern is not intrinsically plausible. For example, if the third dispense had been 15 units instead of 10, the pattern weight for pipi would have been much lower (bubble moved from 10 to 15 on the dotted-grey size curve under pipi). In that case, the algorithm would have failed the Data Confidence test and refrained from labeling the session. This illustrates a design goal of the exemplary algorithm, which is that it should not attempt to label a session if an experienced human interpreter would not be confident doing so.

### Example 2: Bolus Walk-Through

This walk-through covers four sessions (102-105) for user #3821 in an insulin bolus study data set. Because bolus-drug analyses are slightly more complicated than basal, this walk-through builds on the basal walk-through for user #8511 above. That walk-through along with the below detailed description of an exemplary “full” algorithm may be necessary for fully understanding the example.

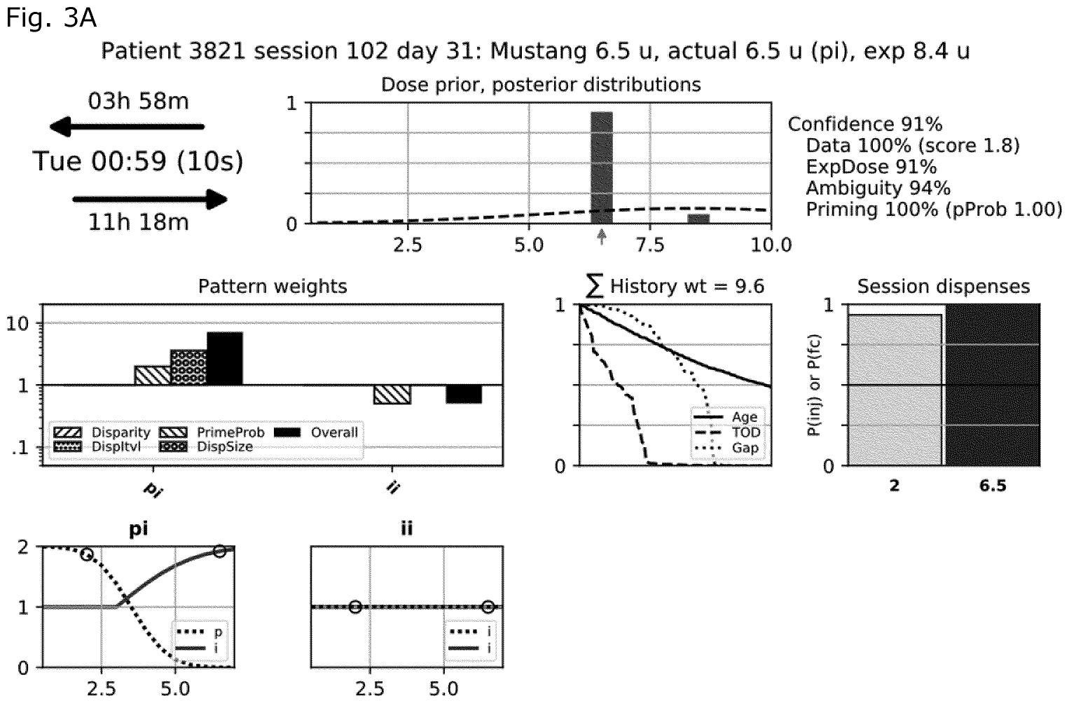

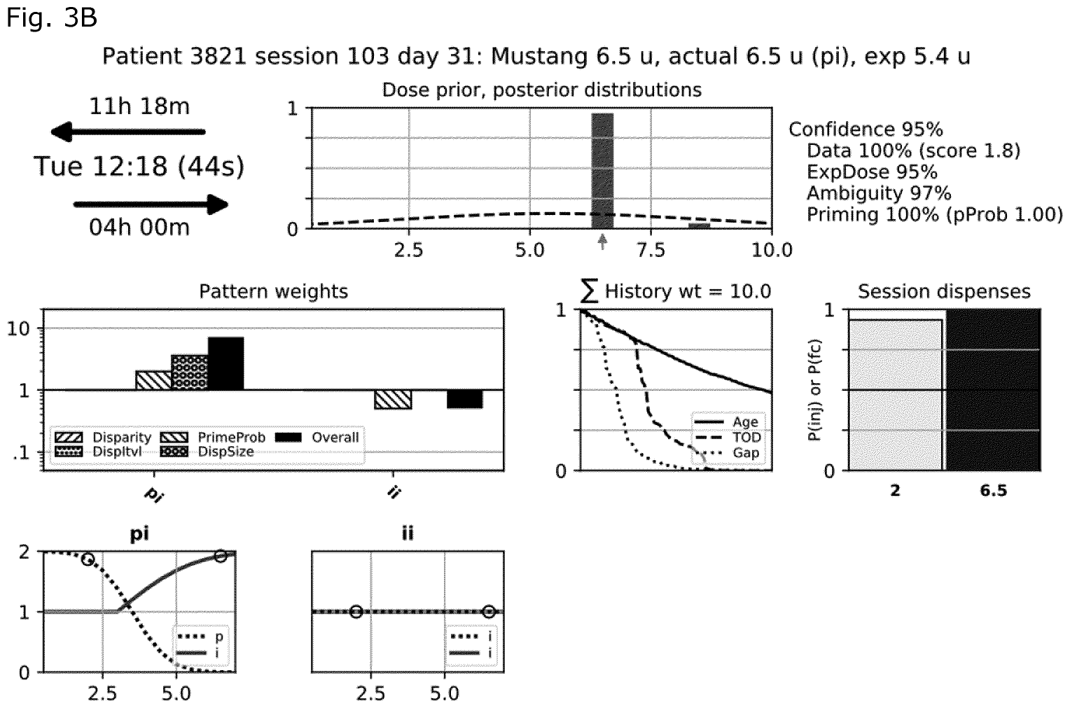

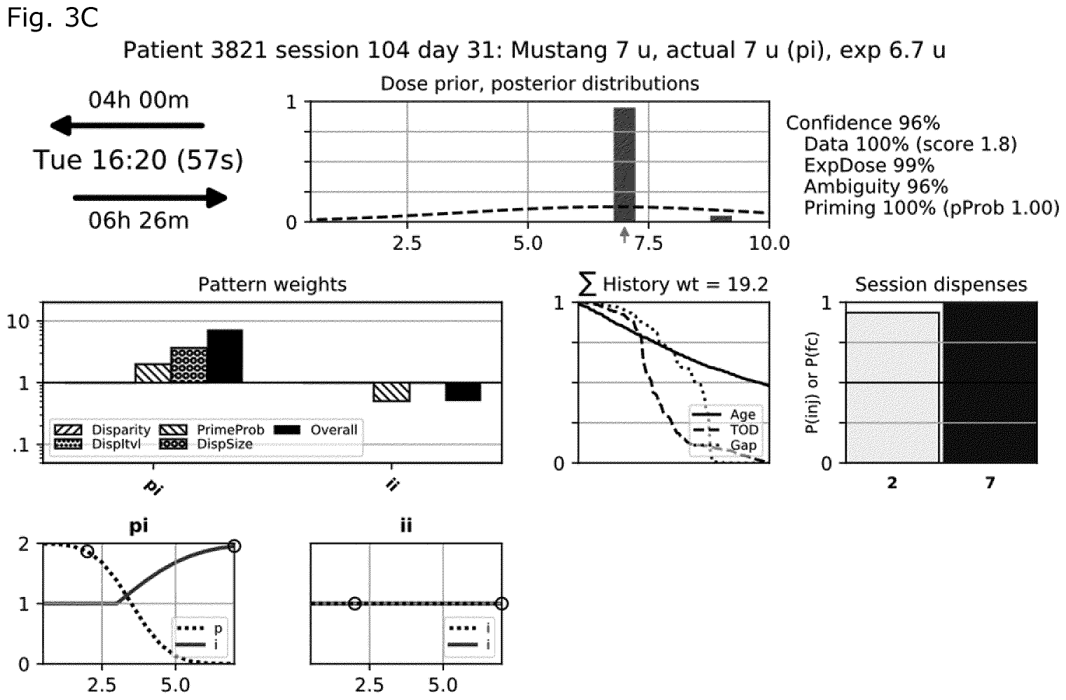

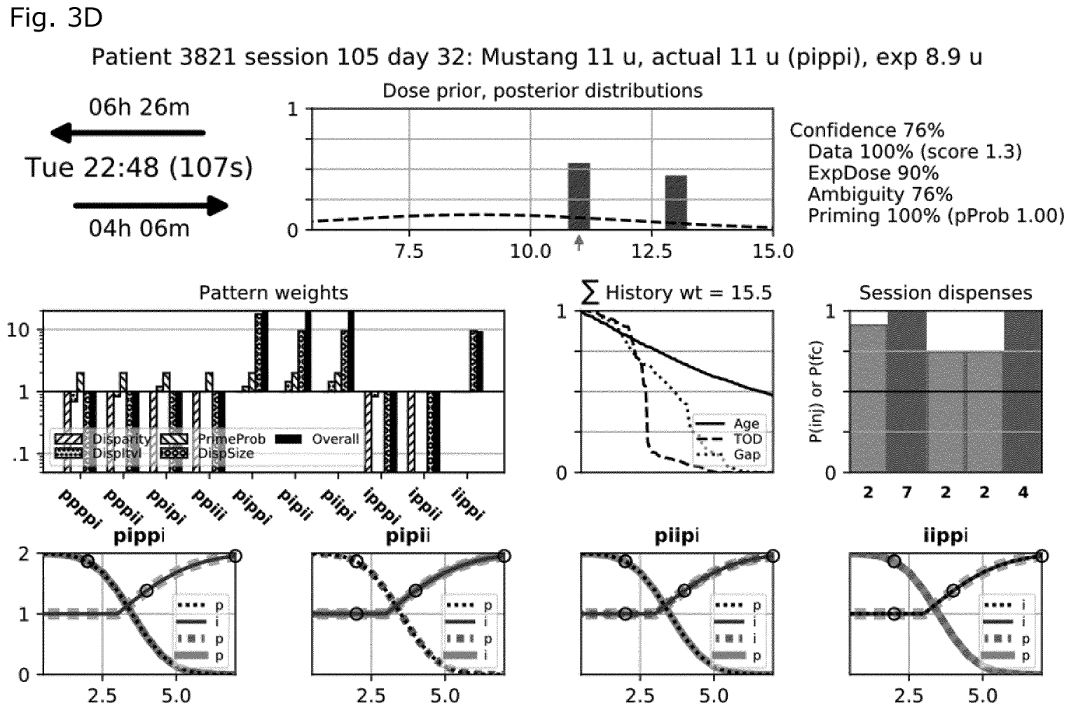

Session 102—FIG. 3A

The session is {2, 6.5} which the user in the study has reported as a prime followed by an injection, and the algorithm came to the same conclusion with high confidence (91%), thus it has labeled the session. An injection of 6.5 units is actually on the high side of what we typically encounter in bolus data, it is not at all uncommon for injections to be one or two units, about the same size as a priming dispense (flow check). This lack of easy differentiation based on the dispense size is what drives most of the differences between the basal and bolus versions of the algorithm.

The allowable patterns are the same as for basal, so for two dispenses the possibilities are pi and ii. Pattern weights are the product of all four weight factors for bolus (priming disparity, intra-session dispense intervals, priming probability, and dispense sizes), but priming disparity has no meaning unless there is more than one prime in one of the possible patterns, so for this session its weight factor is one, thus no bar is visible in the pattern-weights subplot. Likewise, the intra-dispense intervals have no meaning unless there are at least three dispenses in the session, so that weight is also one. Looking at the other two weight factors:

1) The pattern pi involves priming whereas ii does not; this user is a very consistent primer (pProb=1.00), so pi gets a priming weight factor >1 and ii gets a priming weight factor <1 (hatched-down, PrimeProb). This is the same as the basal algorithm.

2) The pattern pi also gets the highest size-based weight factor (DispSize, circle-pattern bars), about 4 judging from the circles in the “pi” plot at lower left (1.9*1.9˜=4). The alternative, ii, gets a sizebased weight factor of exactly one. The weight factor is still computed as the product of samples, indicated by open circles, on the size curves in the bottom row of plots. The curves themselves, however, are different for bolus.

One rule is that candidate injections never receive weights less than one, as seen in the solid curve in the “pi” plot. This is because, while it is still true that large dispenses are more likely to be injections, it is *not* true that small dispenses are more likely to be primes; therefore we cannot down-weight a pattern just because its injection(s) correspond to small dispense(s). Recall that weights greater than one signify “highly likely” and weights less than one signify “unlikely”, a weight of exactly one is neutral.

The size curve for candidate injections will also be shifted depending on the average size of the candidate primes in the pattern (see the below detailed description of an exemplary algorithm for details). The idea is to have the injection curve start rising as early as possible, but only after the size which is being used for priming dispenses.

When there are no candidate primes in a pattern (which is the case for pattern ii in this session), all of the size weights are left at one. We could use a default injection size curve instead, thereby up-weighting the pattern when the dispenses are large, but that would risk giving no-prime patterns an unfair advantage, because the injection curves cannot be less than one. Simply leaving the size weight equal to one in this case was found to be more reliable.

Notice that the Gaussian dose prior distribution (dotted curve in the upper subplot) is very wide. This is characteristic of bolus data, because it turns out that historical doses are a poor predictor of the future. Generally, when all of the historical doses are combined in their weighted average (with weights depending on similarity in time of day, similarity in gap from the last dose, and age of the session), the estimated variance is high, so the Gaussian dose prior is wide. In this case, the prior distribution actually favors the wrong pattern (ii), but the effect is small and not enough to keep the algorithm from the correct answer with high confidence.

Session 103—FIG. 3B

The session analysis is nearly identical to session 102, but the expected dose is different this time, and closer to the actual dose. This session occurred at a completely different time of day (12:18 vs 00:59), and the gap from the previous session is also different (11:18 vs 03:58), so the weighted average in the expected-dose calculation emphasizes a different group of past sessions. Whether this makes the expected dose more accurate, in general, can be discussed. In the absence of outside dose-guidance information, however, going by history is a valid approach.

The more-accurate dose prior, and specifically, the peak of the prior (5.4 units) being on the opposite side of the correct dose from the next-most-probable dose (8.5 units, from pattern ii), means that the dose prior is emphasizing the correct dose at the expense of the incorrect dose. This has reduced the ambiguity between the two, which is reflected in the 97% ambiguity confidence score—compare to session 102.

Session 104—FIG. 3C

This is again similar to sessions 102 and 103, with an even better expected dose (6.7 units vs actual 7 units). The group of past sessions used in the expected-dose weighted average is again different, thanks to the different time of day (16:20 vs 00:59 and 12:18). As evidence of this, notice that the sum of history weights has suddenly almost doubled, to 19.2. This simply means that there were many more “similar” sessions available (on the basis of time-of-day, gap since previous session, and age) for the average. Perhaps this user doses more frequently near 16:00 than at 00:00 or 12:00.

Session 105—FIG. 3D

The dispense pattern {2, 7, 2, 2, 4} appears to be a split dose, likely due to a cartridge change. The larger number of dispenses makes it a good case study in pattern weights.

There are ten different patterns permitted by the algorithm analysis, making the pattern-weights graph a bit tricky to read. Notice first that all five bars are being used—the four weight factors and their product (the overall weight). Also, while there are ten possible patterns, there is only space to show the size-based weighting curves for four of them, so only the top four are shown (corresponding to the highest four solid bars, sorted from highest to lowest overall weight).

1) The “priming disparity” (hatched-up bars, Disparity) was devised to penalize patterns where the candidate primes do not agree. The theory is that a given user has a preferred priming dispense size, in this case it appears to be two units. If the candidate primes in a pattern do not all have the same size, the priming disparity weight factor is set <1 (a penalty), depending on the amount of disparity. (Wrong patterns will naturally have a high disparity, for example ppppi, which helps to down-weight them.) The highest possible disparity weight is simply one, i.e. this weight never gives a boost, only a penalty.

2) The intra-session dispense timing (dotted bars, Displtvl) is moderately helpful. The most likely patterns based on timing alone are pipii and piipi which are incorrect, but pippi scores a close second and the other patterns mostly get a penalty (weight factor less than one).

3) Most of the patterns score well on priming (hatched-down bars, PrimeProb). Only the patterns beginning with an injection (no leading prime) are penalized, due to the user's high priming probability (1.0).

4) The correct pattern gets the highest size-based weight (circle-pattern bars, DispSize), because both 4- and 7-unit dispenses score higher than one on the candidate injection curve. (This curve is big-dotted grey in the leftmost plot in the bottom row; note that a new pattern is automatically assigned for each dispense no matter how many, but big-dotted grey does not appear in the legend because there is only space there for the first four dispenses. The big-dotted grey curve is overlying the solid i-curve.) The other three highly-ranked patterns score lower, but not by much. This is a consequence of never weighting candidate injections less than one; it thus becomes possible to “take” any of the two-unit primes as an injection, with minimal cost to the size-based weight.

The overall pattern weights for the three highest-ranked patterns (pippi, pipii, and piipi) are only a little lower than the weight for the correct pattern. The answer is “saved” in this case by two things:

First, the expected dose (peak of the Gaussian dose prior, dotted curve in top plot) is on the low size of the correct dose, whereas the incorrect doses are on the high side; this penalizes the incorrect doses relative to the correct dose. Second, all of the highest-ranked incorrect patterns result in an [incorrect] dose of 13 units, equivalent to each of the two-unit primes being taken as an injection. This raises the probability of a 13-unit dose (posterior distribution, dark-grey bars), but in cases like this where more than one pattern results in the same dose, the ambiguity is calculated using only the highest-weighted of the possibilities in that dose bin. Thus, the ambiguity confidence score (76%) is higher than might be expected from the posterior probabilities alone.

Dealing with split doses in bolus data is a challenging topic. To further improve identification additional cues may be used, e.g. the user's typical priming-dispense size. It may also be possible to infer the likelihood of a split-dose pattern based on the known capacity of a pen/cartridge and the total amount dispensed since last time a split dose was identified.

Next a detailed description of an exemplary, comprehensive “full” version of an algorithm incorporating all the different aspects and options of the invention will be given.

### 1. Introduction

The algorithm is an algorithm for classifying dispenses from a drug-delivery device (e.g. an insulin pen) as either flow checks (flow check and priming is used synonymously) or injections. It does this using nothing more than the raw dispense data—dispense sizes and timestamps—from the device.

The algorithm consists of inter-related Segmentation, History, and Session Analysis components which work to split the incoming data stream into logical chunks (sessions), estimate an overall dose for each session, and track historical dosing behavior. These components are described in the following sections.

Flow of data through the Flow Check Prediction algorithm can be summarized as: First the data is segmented into sessions, then two parallel analyses are performed: one to analyse the patients past behaviour, the other to analyse the current session. This data is then combined to calculate an estimated dose, then this estimated dose goes through a series of confidence tests before a final determination is given about the session, i.e. the session output.

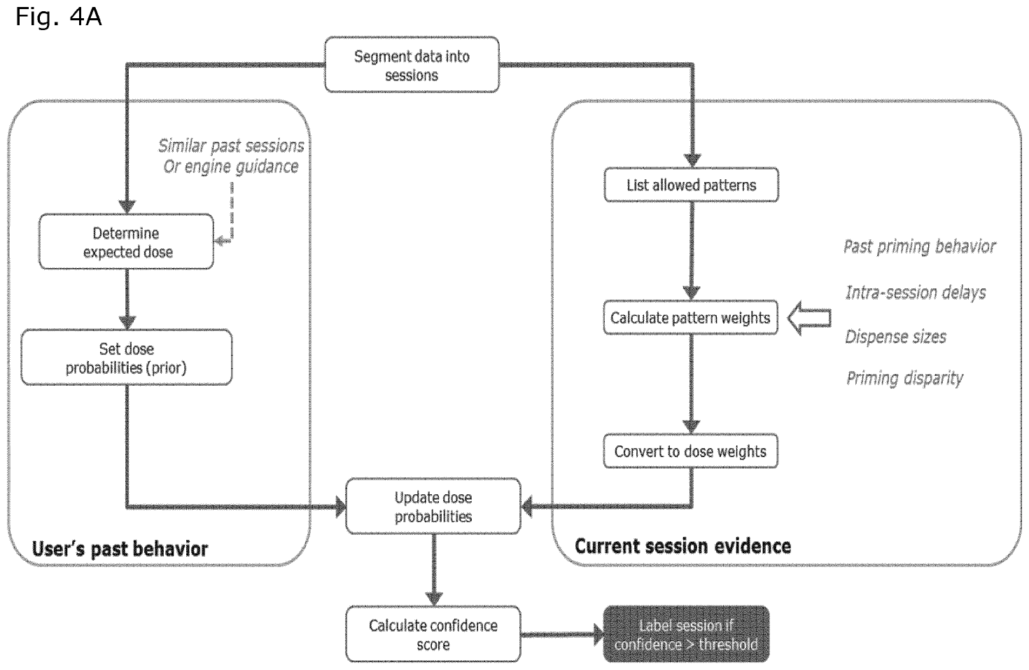

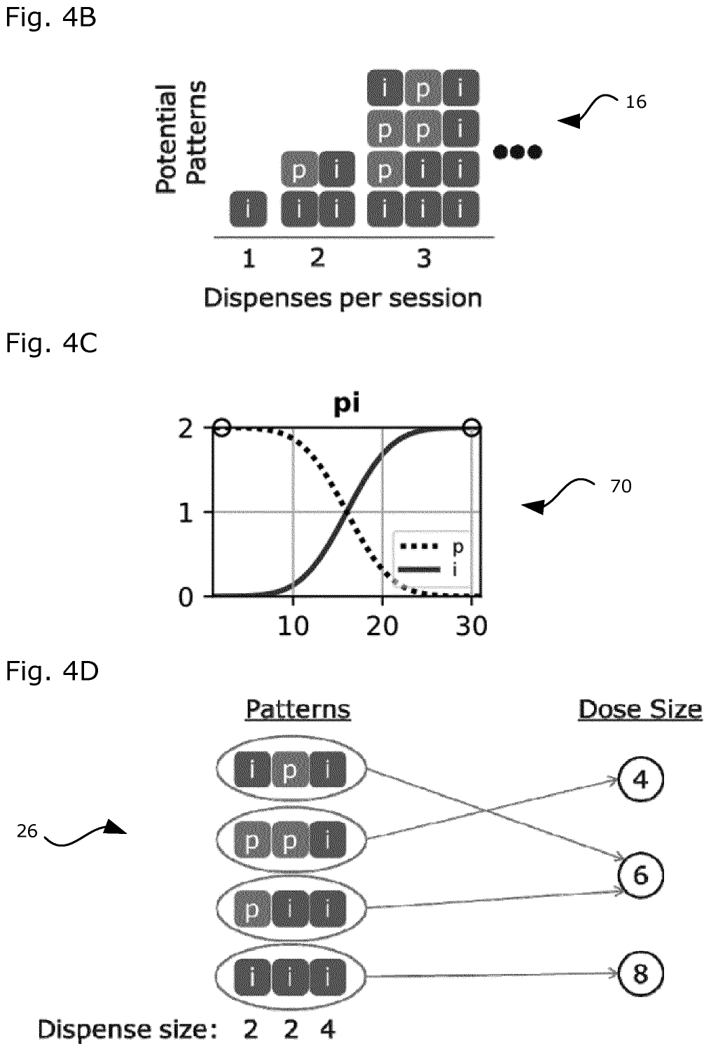

The full version of the algorithm is outlined in FIG. 4A, wherein the “current session evidence” flow chart represents the core part of the algorithm whereas the “user's past behaviour” flow charts represents optional refinements to the core algorithm to further improve precision and reliability.

### 2. Segmentation Component

The principal input to the algorithm is a time-stamped series of dispense records. The segmentation module is responsible for:
grouping dispenses into sessions, which are stored as session objects,
when a session is complete (no more dispenses can be added to it), requesting that session object to perform dose estimation on itself, and
after dose estimation, sending the session summary (dose, classified dispenses, confidence, etc.) to the history module for storage

The segmentation module can be viewed as a “session factory” because it is responsible for creating session objects as needed to hold dispenses, and destroying session objects when they are no longer needed. Each new session object is initialized with its list of dispenses, the elapsed time since the last dispense of the last session (called the “preceding gap”), and a serial number which starts at one for the user's first session.

The lifetime of the segmentation module is equal to the lifetime of a user in the system, so it is the part of the algorithm which interfaces with the higher-level API (Application Program Interface).

The segmentation module provides an “add dispense” method which clients use to notify it of new dispenses. Depending on the nature of the connected pen or other drug-delivery device, these notifications may happen in real time, or in batches at some later time. The only timing requirement is that dispenses must be added to the segmentation module in the same order in which they occurred. Out-of-order dispenses will confuse the segmentation logic.

When a session is complete (as determined by the segmentation algorithm), the segmentation module asks the session object to perform dose estimation on itself, passes the result back to the client, and causes a summary of the session to be saved in the history module.

2.1 Segmentation Algorithm

The algorithm groups dispenses into sessions with a simple clustering criterion using only three configurable parameters. Two time periods, window and windowMax (>=window), start counting from the first dispense of a session, and a ratio, gapRatio, controls the required degree of clustering.

Without loss of generality, consider a session [symbol] ^{k} which has just started with a single dispense d₁^{k} at time t₁^{k}. The last dispense of the previous session [symbol] ^{k-1} is denoted dₗₐₛₜ^{k-1} and happened at time tₗₐₛₜ^{k-1}. The first dispense of the next session [symbol] ^{k+1} will be called d₁^{k+1}, happening at time t₁^{k+1}.

The current session started with dispense d₁^{k}. Subsequent dispenses {dₙ^{k}}_{n=2, 3, . . .} get added to the session if:
A) the session timer<window−that is, dₙ^{k} gets included if tₙ^{k}−t₁^{k}<window,

or
B) the session timer<windowMax and, when dₙ^{k} is assumed to be dₗₐₛₜ^{k}, the resulting session [symbol] ^{k} would satisfy both

tₗₐₛₜ^{k}−t₁^{k}<(t₁^{k}−tₗₐₛₜ^{k-1})/gapRatio and tₗₐₛₜ^{k}−t₁^{k}<(t₁^{k+1}−tₗₐₛₜ^{k})/gapRatio

In other words, dispenses are always included in the current session until window time after the session start. Dispenses may be included up to windowMax time, but only if the gaps between this session and the preceding/following sessions are both longer than gapRatio times the length of this session. As discussed next, dispenses falling between window and windowMax cannot be tested until some time after windowMax has passed.

2.2 Causality and Real-Time Operation

The gapRatio tests imply that when there are provisional dispenses, it is not possible to know a session is complete until some time after the fact. Dispenses arriving between window and windowMax may, or may not, become part of [symbol] ^{k}.

Their membership is uncertain, and [symbol] ^{k} remains open, until either:

1) Enough time passes that the next dispense is guaranteed to meet condition (B) above (in which case the provisional dispenses all stay in [symbol] ^{k}). If we assume the previous session gap test from condition (B) is met, a variable “new session threshold” can be calculated as:

t_{newSession}=tₗₐₛₜ^{k}+gapRatio*(tₗₐₛₜ^{k}−t₁^{k})

If a new dispense comes in after this new session threshold, tₗₐₛₜ is included in the current session and the new dispense defines the first dispense in a new session.

or

2) Another dispense arrives, after windowMax but before the new session threshold defined above. As soon as this happens, the session membership of all the potential dispenses in the current session is resolved, some becoming part of the current session or part of [symbol] ^{k+1}.

First, assume that all of the dispenses prior to windowMax belong to [symbol] ^{k} and the new dispense is t₁^{k+1} for the next new session [symbol] ^{k+1}. Condition (B) should fail (because the new session threshold has not yet been reached), so push tₗₐₛₜ^{k} out of [symbol] ^{k} and into [symbol] ^{k+1} and make it the new first dispense of the new session and retest condition (B) from above. Continue in this way until either (1) a dividing point has been found between [symbol] ^{k} and [symbol] ^{k+1} which satisfies condition (B), or (2) all of the provisional dispenses have been taken out of [symbol] ^{k}.

At this point, all of the dispenses which were taken out, together with the new dispense (the one outside of windowMax), should be processed as [symbol] ^{k+1} with the session timer now starting at whichever dispense ended up as d₁^{k+1}. (Note that in rare cases, it is conceivable that this list of dispenses would itself be split between [symbol] ^{k+1} and [symbol] ^{k+2}, etc. recursively.)

In some usage scenarios, a pen communicates dispenses to the algorithm in near-real-time and client software may desire user feedback after dose estimation. For example, when confidence is low and the algorithm chooses not to label the session, the user may be asked to classify the dispenses manually as flow checks or injections. In such cases, the worst-case latency of the segmentation algorithm needs to be considered. By definition, the longest possible session has a duration equal to windowMax, therefore, condition (B) will always be met after at most windowMax×gapRatio from the last dispense. This is the worst-case waiting time until [symbol] ^{k} is known to be complete and dose estimation can proceed. At the current values of these parameters for basal- and bolus-insulin datasets, the worst-case wait from the last dispense is 2½ hours for basal and 35 minutes for bolus.

If this much latency cannot be tolerated by the application, it is possible to set windowMax=window which simplifies the algorithm to condition (A) only, at the possible expense of segmentation accuracy. Note, however, that it is relatively rare for dispenses to occur between window and windowMax, so in practice the typical latency is windowMax from the start of the session, much lower than the worst-case. (windowMax is currently 30 minutes for basal and 7 minutes for bolus.)

2.3 Segmentation Component Parameters

| | | Typical |
|---|---|---|
| Parameter | Description | value |
| window | A group of dispenses within a window of this | 360 sec |
| | duration will always count as a single session. | (basal), |
| | | 300 sec |
| | | (bolus) |
| windowMax | Sessions are allowed to extend up to | 1800 sec |
| | windowMax if the time gaps on each side of | (basal), |
| | the resulting session are at least gapRatio | 420 sec |
| | times the session length. | (bolus) |
| gapRatio | (See windowMax.) | 5.0 |

### 3. History Component

The history module maintains a list of all past sessions for the current user, and tracks statistics which enhance the overall algorithm's classification accuracy. After each session is complete and a labeling decision has been made, the segmentation module provides the history module with the following summary data for storage:
Session timestamp (taken as the timestamp of the last dispense in the session)
Inter-session gap (time from last dispense of previous session to first dispense of this session)
Estimated dose
Was the session labeled? (true/false)
Did the user perform flow checks? (true/false, defined as at least one flow check during the session)
A list of the dispenses comprising the session in the form of (x, y) pairs where x is the dispense size and y is a Boolean variable which is true for an injection and false for a flow check

The history module provides a method for the segmentation module to call to provide this information. Upon request, the history module provides the following statistics to clients: Average sizes of flow-checks and injected dispenses, the user's “priming probability”, and Expected-dose mean and variance.

3.1 Average Sizes of Flow-Check and Injected Dispenses

These are averages over all of the user's recorded dispenses (in all past sessions). The flow-check average size is taken over all dispenses classified as flow checks, and the injected average size is taken over all dispenses classified as injections. Only dispenses from labeled sessions are counted.

Both averages are weighted with a decay factor exp(−t ln(2)/sizeTimeHalf) so that recent dispenses are weighted more strongly. sizeTimeHalf is a configurable parameter and the age t is computed from the session timestamps stored within the history module, thus all of the dispenses in a session end up with the same t. The formula for the average is:

$$\frac{{\sum }_{k} exp ( - \frac{{t}_{k} ln ( 2 )}{s izeTimeHalf} ) {d}_{k}}{{\sum }_{k} exp ( - \frac{{t}_{k} ln ( 2 )}{s izeTimeHalf} )}$$

where the {dₖ} are the dispense sizes (labeled sessions only).

If the sum of the weights (the denominator in the equation above) is less than the configurable parameter minWeightSum, default values for the two averages are returned in place of the actual average. These default values are also configurable parameters.

3.2 the User's “Priming Probability”

This is a measure of the user's likelihood of priming (defined as performing at least one flow check in a session), and ranges between 0 and 1. It is computed as an average over all past sessions, including unlabeled sessions. Two weight sums, “priming weights” and “non-priming weights,” are computed as follows:

$$w = {\sum }_{k} exp ( - \frac{{t}_{k} ln ( 2 )}{p r imeTimeHalf} )$$

where primeTimeHalf is a configurable decay constant and the {tₖ} are the ages of the sessions with priming (for the priming weight sum) or the sessions without priming (for the non-priming weight sum). The priming probability is then computed as:

$$P [ prime ] = \frac{{w}_{p}}{{w}_{p} + {w}_{n p}}$$

If the denominator (the sum of both weight sums) is less than the configurable parameter minWeightSum, a value of 0.5 is returned instead, signifying maximum uncertainty.

3.3 Expected-Dose Mean and Variance

The history module will generate an expected dose value, represented by a Gaussian probability distribution function (pdf) which represents the user's average dose size and dose variability based on the user's prior dose history. Only labelled sessions (sessions with sufficiently high confidence scores) will be used in the calculation of the expected dose mean and variance. The mean is the expected dose and the variance is inversely proportional to the consistency of the user's past doses.

The computation is similar to the one for average dispense sizes, but it produces a weighted sample variance in addition to the weighted average, and the weighting factors are more complex. Each weight is actually the product of three factors, for time (age), time-of-day similarity, and inter-session gap similarity. The goal is to weight past sessions in proportion to their relevance to the current session, i.e. sessions in the recent past are more relevant than older sessions, sessions which occurred at the same time of day are more relevant, and sessions which followed a session gap of similar duration to the gap between the current session and its immediate predecessor are more relevant. The weight formulas are:

$$ageWeight = exp ( - \frac{{t}_{k} ln ( 2 )}{t i m eHalf} )$$

$$timeOfDayWeights = exp ( - ln ( 2 ) {( \frac{{TOD}_{diff , k}}{TODHalf} )}^{2} )$$

$$gapWeights = exp ( - ln ( 2 ) {( \frac{g a {p}_{diff , k}}{g a pHalf} )}^{2} )$$

$$historyWeights = ageWeights * timeOfDayWeights * gapWeights$$

where for each past session k, tₖ is the session age, TOD_{diff,k} is the difference in time-of-day between that session and the current one, and gap_{diff,k} is the difference in inter-session gap between that session and the current one. Note that TOD_{diff} must be computed correctly in a circular fashion, so that, e.g. the TOD difference between 01.00 and 23.00 is two hours, not twenty-two hours. One way of ensuring this, for two variables in “seconds from epoch” format, is:

TOD_{diff}=min((time₁−time₂)mod 86400,(time₂−time₁)mod 86400)

The decay factor timeHalf and the tightness factors TODHalf and gapHalf are individually configurable parameters with the same units of time as the time quantities in the numerators of the exponentials.

If ΣhistoryWeights<minWeightSum, there is insufficient history to make a good calculation and the history module will refuse to provide the expected dose parameters, instead setting the mean to zero and the variance to minVariance (see below). Otherwise, the expected-dose mean and variance are computed as:

$$μ = \frac{{\sum }_{k} ( h i s t oryWeig h t s * {d}_{k} )}{{\sum }_{k} h i s toryWei g h t s}$$

$${σ}^{2} = max ( minVariance , \frac{{\sum }_{k} ( h i s t oryWeig h t s * {( {d}_{k} - μ )}^{2} )}{{\sum }_{k} h i s t oryWeig h t s} )$$

where the {dₖ} are the session doses for all past labeled sessions, each with their corresponding history weight. The variance (σ²) is constrained to always be ≥minVariance (a configurable parameter). In effect, this keeps the algorithm from being too certain about the expected dose when a user has been extremely consistent with one dose in the past.

3.4 History Component Parameters

| | | Typical |
|---|---|---|
| Parameter | Description | value |
| timeHalf | Exponential decay rate for discounting session age (more | 10 days |
| | recent sessions count more than older sessions) in the | (basal), |
| | expected-dose calculation. The weight is 50% after this | 28 days |
| | time. | (bolus) |
| TODHalf | Gaussian decay half-width for similarity in time-of-day in | 3 hours |
| | the expected-dose calculation. (Past sessions occurring | |
| | at the same time of day as the current session count more | |
| | than sessions at other times of day.) The weight is 50% | |
| | at +/− this time. | |
| gapHalf | Gaussian decay half-width for similarity in preceding gap | 10⁶ sec |
| | in the expected-dose calculation. (Past sessions which | (basal), |
| | were preceded by a gap of similar length as the current | 2½ hours |
| | session count more than sessions with longer or shorter | (bolus) |
| | preceding gaps.) The weight is 50% at +/− this time. | |
| minWeightSum | The history-based expected dose is only calculated when | 3.0 |
| | the sum of all of the weights exceeds this value. (When | |
| | there is no expected dose, the dose prior becomes uniform | |
| | and the expected-dose confidence is automatically | |
| | 100%.) minWeightSum can be viewed as the number of | |
| | past sessions required, if their weights were all one. Due | |
| | to the age/TOD/gap weighting, the past sessions weights | |
| | are always substantially less than one, so more sessions | |
| | than this are required in the average. | |
| minVariance | The history-based expected dose calculation also provides | 4.0 |
| | the expected statistical variance of its answer. This | (basal), |
| | controls the width of the Gaussian dose prior distribution. | 10.0 |
| | There is a danger that if a user is very consistent in dosing | (bolus) |
| | for a long time, then needs to change their dose, the | |
| | algorithm could start rejecting the new dose because it is too | |
| | far away from the expected dose (based on history). | |
| | minVariance places a floor on the variance to mitigate this | |
| | risk. | |
| primeTimeHalf | Exponential decay rate for discounting session age (more | 14 days |
| | recent sessions count more than older sessions) in the | |
| | primeProb (priming probability) calculation. The weight is | |
| | 50% after this time. | |
| sizeTimeHalf | Exponential decay rate for discounting session age (more | 14 days |
| | recent sessions count more than older sessions) in the | |
| | average flow-check/injection size tracking. The weight is | |
| | 50% after this time. | |
| defaultAvgFCSize | Default average dispense size for flow checks (primes), | 2.0 |
| | before there is enough history to calculate the average | |
| | based on that. | |
| defaultAvgInjSize | Default average dispense size for injections, before there | 9.0 |
| | is enough history to calculate the average based on that. | (basal), |
| | | 5.0 |
| | | (bolus) |

### 4. Session Analysis Algorithm

Session objects are containers for dispenses, which are grouped there by the segmentation module. Each session object also contains the methods (code) required to analyse its dispenses, decide on the most likely session dose, and rate its confidence in the result. The session module's methods can be divided into three groups.

4.1 Adding and Removing Dispenses

The session module provides methods for appending a dispense to the end of the session, or removing a dispense from the end of the session and returning it to the caller. Both methods are used by the segmentation module.

4.2 Helpers

The session module provides utility methods to report
the session duration (the timestamp difference between the first and last dispenses of the session).
whether the session is labeled or not. (The session is labeled if and only if it has been finalized and all of the confidence scores are greater than confidenceThreshold.)
the confidence measure resulting in the lowest confidence score, whether or not the session was labeled.
the pattern of primes and injections which best explained the session (called the “winning pattern”).

4.3 Session Analysis and Dose Estimation

Dose estimation is the core functionality of the session module. The analysis can be divided into four phases as shown in the figure below: First the algorithm enumerates all of the patterns of primes (flow checks) and injections which might be used to interpret the session dispenses, then it weights those patterns according to various criteria, next it converts the weighted patterns into weighted dose estimates and uses Bayes rule to combine them with a dose prior (based on the user's dosing history), obtaining the dose posterior and the most-likely session dose, finally, it applies confidence checks which determine whether or not the session will be labeled.

These phases correspond to the methods enumerate_patterns, weight_patterns, estimate_dose, and evaluate_result in the reference implementation. Each will now be described in detail.

4.3.1 Pattern Enumeration

Given a session with N_{disp} dispenses, the dose-estimation problem is entirely equivalent to the pattern-selection problem: After each dispense is classified as either a flow check (often referred to as a “prime” for brevity) or an injection, the resulting pattern implies the session dose. Using p and i as shorthand, a session with dispenses {2, 1, 2, 4} has a dose of 4 if the pattern is pppi, 5 if the pattern is pipi, or 6 if the pattern is ppii.

Pattern enumeration is the task of listing all potential patterns 16 (FIG. 4B) given some simple rules and configurable parameters:
The last dispense of the session must be an injection.
Each injection must be preceded by at least minPrimes flow checks. This is normally set to zero so that flow checks are not strictly required, otherwise the labeling rate will be extremely low.
The number of injections in a session is limited to maxInjectsSimple plus an allowance for large doses detailed below.

The following pseudocode will perform pattern enumeration:

```
Given N_{disp} (number of dispenses in the session), dialMax (the largest
dispense that can be dialed on the drug-delivery device), and
the configurable parameters maxInjectsSimple and minPrimes,
$maxInjects = min ( {N}_{disp} , maxInjectsSimple + ⌊ \frac{\sum dispenses}{dialMax} ⌋ )$
(Note the “floor” operator)
Initialize P to an empty list of patterns.
for Nᵢₙⱼ = 1 to min(maxInjects, N_{disp} − minPrimes)
append to P the pattern having N_{disp} − Nᵢₙⱼ primes followed by
Nᵢₙⱼ injections
for Nᵢₙⱼ = 2 to maxlnjects
for Nᵢₙⱼ_before = 1 to Nᵢₙⱼ − 1
  for N_{leading}_primes = minPrimes to N_{disp} − Nᵢₙⱼ
    if N_{disp} − Nᵢₙⱼ − N_{leading}_primes ≥ minPrimes
      append to P the pattern having N_{leading} _primes primes
      followed by Nᵢₙⱼ_before injections
      followed by N_{disp} − N_{inj - Nleading}_primes primes
      followed by Nᵢₙⱼ − Nᵢₙⱼ_before injections
Scan through P and remove any duplicate patterns.
```

4.3.2 Pattern Weighting

Each pattern receives a weight which is the product of many independent weight factors:

patternWeight[pattern]=ΠweightFactor[pattern]

The larger the weight, the more likely that that pattern is the correct one. Weight factors are derived from the timing and sizes of dispenses, the priming behavior of the user, and other criteria. Some apply only for a certain drug type, others only to sessions with some minimum number of dispenses. The following subsections describe a concrete example for how each of the weight factors can be determined.

Note that each weight factor is calculated multiple times, once for each pattern in P. In this context it is often necessary to refer to dispenses as flow checks or injections, these classifications refer to the pattern being evaluated, not actual truth.

4.3.2.1 Intra-Session Dispense Intervals

Statistical analysis has revealed that the time between two consecutive dispenses is longer when a user is switching from priming to injecting, versus continuing a series of flow checks.

In the datasets studied, the critical interval is about 3.5 seconds—longer than this and the two dispenses are probably pi (flow checks followed by injection), shorter and the dispenses are probably pp (two flow checks). A weight factor exploiting this tendency is applied for sessions with N_{disp}≥3. (When N_{disp}=2, the second dispense is always an injection so this has no predictive power.) Pseudocode:

if N_{disp} ≥ 3

N_{pp,short} = # of pp occurrences with time interval < criticalDispenseInterval
N_{pi,long} = # of pi occurrences with time interval ≥ criticalDispenseInterval
N_{pi,short} = # of pi occurrences with time interval < criticalDispenseInterval
N_{pp,long} = # of pp occurrences with time interval ≥ criticalDispenseInterval
weightFactor = dispenseIntervalFactor^{(N}^{_{pp,short}}⁺^{N}^{_{pi,long}}⁻^{N}^{_{pi,short}}⁻^{N}^{_{pp,long}}⁾

else

weightFactor = 1

For example, if N_{disp}=4 with dispense timestamps 0, 2, 7, and 10 seconds, the configurable parameters criticalDispenselnterval=3.5 seconds and dispenselntervalFactor=2, the weight factors for patterns pppi, ppii, and pipi would be:
pppi: 2⁽¹⁺⁰⁻¹⁻¹⁾=0.5
ppii: 2⁽¹⁺¹⁻⁰⁻⁰⁾=4.0
pipi: 2⁽¹⁺¹⁻¹⁻⁰⁾=1.0

4.3.2.2 Priming Probability

If a user has performed flow checks regularly in the past, the patterns being evaluated in the current session should be weighted to skew in favor of consistency, that is, patterns with priming should be weighted higher than patterns without. Conversely, if the user has consistently failed to perform a flow check in the past, the patterns should be weighted in the opposite direction, so that patterns without priming are considered more likely. The history module provides a “priming probability” primeProb which ranges between 0 (never primes) and 1 (always primes). A configurable parameter primeProbFactor controls the relative strength of this weight factor.

For the purpose of this calculation, a “pattern with priming” is actually defined as a pattern which begins with a prime, so pi is a pattern with priming, ii is a pattern without priming, but ipi is considered neither. (Patterns with priming between two injections are associated with cartridge changes. New-cartridge priming is somewhat different than routine pre-injection priming, so the weight factor is intentionally unaffected by this. Note however, that pipi, also a likely cartridge change, would still be considered a “pattern with priming” due to its leading prime.)

Pseudocode:

if the first dispense of the pattern is a prime

weightFactor = primeProbFactor⁽²*^{primeProb-1)}

else if all of the dispenses in the pattern are injections

weightFactor = primeProbFactor⁽¹⁻²*^{primeProb)}

else

weightFactor = 1

4.3.2.3 Priming Disparity (Bolus Drugs Only)

When attempting to distinguish primes from the small injections which are common in bolus-drug patients, it can be helpful to down-weight patterns where the priming dispenses do not have a consistent size. This is the idea behind priming disparity, with configurable parameter bolusPrimeDisparityFactor:

Given a list called primes with the dispense sizes for all of the primes in
the pattern being evaluated,
if N_{disp} ≥ 3 and Nₚᵣᵢₘₑₛ ≥ 2 for this pattern

disparity = max(primes) − min(primes)
weightFactor = bolusPrimeDisparityFactor^{-disparity}

else

weightFactor = 1

4.3.2.4 Dispense Size (Bolus Drugs)

Dispense size is perhaps the most obvious differentiator between primes and injections: the simplified view is that primes are small and injections are large. This does not work well for patients on MDI (basal/bolus) therapy, however, because typical bolus doses can easily be as small as the recommended priming size of two units. The priming size distribution is expected to peak near two units, but the injection size distribution does not give much insight, except that very large dispenses are likely to be injections.

For both basal and bolus sessions, the size-based weight factor is the product of a series of “size factors” one for each dispense in the pattern. The size factors are samples from shifted and scaled copies of the cumulative Gaussian function 70,

$$erf ( x ) = \frac{2}{\sqrt{π}} {\int }_{0}^{x} {e}^{- {t}^{2}} dt$$

There is no deep reason why erf( ) is used, it is merely a convenient and widely available S-shaped function. A smooth S curve models threshold behavior (for example, “above y units probably an injection, below y units probably a prime”) without introducing discontinuities in algorithm behavior at special dispense sizes, see fig. XX. The transition area can be narrowed or widened as needed by scaling the operand, and the asymptotic values of the function are also easy to modify. In the present usage, 1+erf( . . . ) and 1−erf( . . . ) expressions yield outputs ranging from 0 to 2, which is convenient for constructing a weight factor. At the center of the S curve the output will be 1, which is neutral in the weight factor, see FIG. 4C.

Pseudocode:

```
Given two lists - primes and injects - with the dispense sizes for the flow
checks and injections in the pattern being evaluated, and the configurable
parameters bolusPrimeSizeSlope,bolusPrimeCrossoverSize,
bolusInjectSizeSlope, and bolusInjectSizeOffset, weightFactor = 1
if Nₚᵣᵢₘₑₛ > 0
    $avgPrime = \frac{\sum primes}{{N}_{primes}}$
    for x in primes
        weightFactor = weightFactor *
      (1 − erf (bolusPrimeSizeSlope * \sqrt{π} * (x −
      bolusPrimeCrossoverSize)))
    for x in injects
        weightFactor = weightFactor *
  max (1, 1 + erf (bolusInjectSizeSlope * \sqrt{π} * (x − avgPrime −
  bolusInjectSizeOffset)))
```

Explanation:

As mentioned earlier, the weight factor is the product of individual size factors which are samples from scaled and shifted S curves. (The weight factor is initialized to one, so the order of computation can be rearranged as desired, here it is shown as separate loops over the list of flow checks and injections, because the size factors are different for each.) For primes the S curve goes from 2 to 0, touching 1 at bolusPrimeCrossoverSize. For injections the S curve goes from 0 to 2, but there are two modifications: First, it can never go below 1. This reflects the fact that bolus injections may be arbitrarily small, and it does not make sense to score them lower just because they overlap with the region of typical primes. Second, instead of a fixed crossover size, the curve has a data-dependent crossover at avgPrime+bolusInjectSizeOffset. (bolusInjectSizeOffset is typically 1.) This adaptivity both allows candidate injections to score higher when the priming size is small, and acts to spoil the score of a candidate injection when a wrong pattern is being evaluated (because avgPrime, being the average of the candidate primes for this pattern, may get larger when some of the candidate primes are actually injections).

4.3.2.5 Dispense Size (Basal Drugs)

For basal-drug sessions, the distinction between primes and injections based on size is somewhat easier because injections tend to be large. To maximize the effectiveness of this discrimination, the algorithm uses the historical average prime and injection sizes and attempts to set an optimal crossover point on the S curve (the size where the weight will be one, i.e. neutral).

Pseudocode:

```
Given two lists - primes and injects - with the dispense sizes for the flow checks and injections in
the pattern being evaluated, a history- or guidance-based expectedDose (zero if there is insufficient
history), historical average dispense sizes historicalPrime and historicalInject, a small number
ϵ to prevent division by zero, and configurable parameters basalSizeSlopeMin,
basalSizeSlopeMax, and basalSplitDoseRatioMin,
weightFactor = 1
if expectedDose > 0
  expDose = expectedDose
else
  expDose = max(historicalInject, max(all dispenses in session))
$primeCenter \frac{( \frac{e x p D o s e}{{N}_{injects}} + historical P r i m e )}{2}$
$\begin{matrix}primeSlope = min ( basalSizeSlopeMax , max ( basalSizeSlopeMin , \\ \frac{1}{\frac{expDose}{{N}_{injects}} - historicalPrime + ϵ} ) )\end{matrix}$
for x in primes
  weightFactor = weightFactor * (1 − erf (primeSlope * \sqrt{π} * (x − primeCenter)))
for x in injects
$\begin{matrix}injectCenter = \frac{\begin{matrix}max ( basalSplitDoseRatioMin \frac{x}{\sum injects} ) * \\ expDose + historicalP r ime\end{matrix}}{2} \\ injectSlope = \\ \begin{matrix}min ( basalSizeSlopeMax , max ( basalSizeSlopeMin , \\ \frac{1}{\begin{matrix}max ( basalSplitDoseRatioMin , \frac{x}{\sum injects} ) * \\ expDose + historicalPrime + ϵ\end{matrix}} ) )\end{matrix} \\ weightFactor = weightFactor * ( 1 + erf ( injectSlope * \sqrt{π} * ( x - injectCenter ) )\end{matrix}$
```

Explanation:

The basic structure of these calculations is similar to that for bolus drugs, in that the size factors are still samples of shifted and scaled S-curves. The bolus formulas, however, make a serious attempt to place the crossover dispense sizes, primeCenter and injectCenter, halfway between the likely prime and injection sizes. Note that expDose is an alias for the history-based expected dose expectedDose, if it exists, otherwise, a stand-in is used instead.

The likely prime size is simply the user's historical average prime. The likely injection size must account for split doses. If the pattern being evaluated has more than one injection,

$$\frac{expDose}{{N}_{injects}}$$

approximates the injection size under the assumption of an even split. This (primeCenter) is good enough for evaluating candidate primes, but it can cause candidate injections to be scored too low when a dose is unequally split, thus the calculation for injectCenter uses the quantity

$$\frac{x * expDos e}{\sum injects} ,$$

replacing

$$\frac{1}{{N}_{injects}} with \frac{x}{\sum injects} .$$

This way, when smaller dispenses x are evaluated as possible injections, the “likely injection size” is lowered, keeping their score high even when the dose is unequally split. This must be limited to prevent, e.g., primes from scoring high as candidate injections, so the parameter basalSplitDoseRatioMin sets a limit on how wide the split can be. (A typical value is 0.2.) Note the position of the injectCenter and injectSlope calculations inside the for loop, because they depend on the size x of each candidate injection in the pattern.

The primeSlope and injectionSlope calculations appear more complicated, but they are built on the same expressions already described. The slope of the S curve should be steeper when the likely prime and likely injection sizes are close together, and shallower when they are further apart. The slopes are just the reciprocal of the difference in these sizes, rather than the average of these sizes used in the center calculations. They are further constrained within upper and lower bounds with two more parameters, basalSizeSlopeMin and basalSizeSlopeMax.

4.3.3 Dose Estimation and Session Interpretation

After computing the pattern weights, the algorithm converts them from a weighting over pattern to a weighting over dose. The mapping from patterns to doses is many-to-one: each pattern results in one dose, but multiple patterns 26 may result in the same dose, see FIG. 4D.

Pseudocode:

initialize doseWeight[dose] = 0 for every dose (0 to capacityAvg in steps
of dialInc)
for each pattern in P

dose = Σ injected dispenses in pattern
doseWeight[dose] = doseWeight[dose] + patternWeight[pattern]

If normalized to sum to one, doseWeight is a probability distribution over
dose.

Another probability distribution over dose is based on the expected-dose mean and variance from the History module. (The expected dose is computed from the user's dosing history.) This is known as the dose prior distribution. It is Gaussian (normal) with mean and variance equal to the expected-dose mean μ and variance σ², or, if there was not enough history to compute an expected dose, a uniform distribution:

```
Given the expected-dose mean μ and variance σ²,
if μ > 0
  for dose = 0 to capacityAvg in steps of diallnc
    $dosePrior [ dose ] = \frac{1}{\sqrt{2 π {σ}^{2}}} exp ( - \frac{{( dose - μ )}^{2}}{2 {σ}^{2}} )$
else
  for dose = 0 to capacityAvg in steps of diallnc
    $dosePrior [ dose ] = \frac{1}{{ }^{( \frac{capacityAvg}{dialInc} ) + 1}}$
```

Now the dose prior distribution and the dose weights can be combined using Bayes' rule to obtain the dose posterior distribution:

for dose=0 to capacity of drug-delivery device, in steps of minimum dial increment

$$dosePosterior [ dose ] = \frac{dose P rior [ dose ] doseWeight [ dose ]}{{\sum }_{d o s e} dosePrior [ dose ] doseWeight [ dose ]}$$

The maximum-likelihood dose (often called the estimated dose, not to be confused with the expected dose) is the dose with the highest probability in dosePosterior:

$$estimatedDose = arg {max}_{dose} dosePosterior \\ ( arg {max}_{dose} f ( dose )$$

means to find the value of dose which maximizes f(dose) and return that value of dose. Thus we are finding the dose size corresponding to the peak of dosePosterior. This is the algorithm's best guess at the true injected dose for the current session.)

Working backwards from the estimated dose, it implies the existence of a “winning pattern”—the pattern of flow checks and injections which yields a dose equal to the estimated dose. (This is not necessarily the pattern with the highest weight, because the dose prior may skew the result.) If only one pattern produced the estimated dose, it is the winning pattern. If more than one pattern yielded the estimated dose, the winner is the one with the highest pattern weight. If more than one of these patterns have the same [highest] weight, and that weight is the highest, the choice is arbitrary.

4.3.4 Confidence Checks and Result Evaluation

The algorithm produces an estimated dose for every session, whether that session is “easy” or “hard.” In the envisioned applications, however, it is usually preferable not to label a session (provide an official dose estimate) unless there is reasonable confidence in the result. The purpose of this fourth and final piece of the Session module is to quantify how confident the algorithm is in the estimated dose.

The algorithm has five independent measures of confidence which can range from 0 (no confidence) to 1 (100% confident), each of these has a configurable weight between 0 and 1:

4.3.4.1 Data Confidence

This depends on the “data score” which is simply the dose weight of the most-likely dose, normalized for varying session length:

$$dataScore = \frac{doseWeight [ estimatedDose ]}{{2}^{{N}_{disp}}}$$

$$dataCinfidence = 1 - ( dataConfidenceWeight \times ( 1 - min ( 1 , dataScore ) ) )$$

A low data confidence indicates that the winning pattern is a poor explanation for the observed session, and was chosen only because the other patterns were even worse.

4.3.4.2 Expected-Dose Confidence

This is another way of using the history-based expected dose as a cross-check on the estimated dose. (The other way is via the dose prior distribution.) It measures how much the estimated dose differs from the expected dose, in standard deviations:

```
Given the expected-dose mean μ and variance σ²,
if μ > 0
  $\begin{matrix}expectedDoseConfidence = \\ max ( 0 , 1 - expectedDoseConfidenceWeight \times ( \frac{abs ( estimatedDose - μ )}{\sqrt{{σ}^{2}}} )\end{matrix}$
else
  expectedDoseConfidence = 1
```

4.3.4.3 Ambiguity Confidence

This penalizes an estimated dose if it does not dominate the second-choice, third-choice, etc. answers by a sufficient margin:

$$ambiguityConfidence = max ( 0 , 1 - \frac{a m b i g u ityConf i d e n ceWeigh t \times ( 1 - max ( d osePost e r i o r ) )}{max ( d osePost e r i o r ) + ϵ} )$$

where ϵ is a small number to protect against division by zero.

4.3.4.4 Priming Confidence

This measures the consistency between the user's past priming history—use of flow checks (as given by primeProb, provided by the history module) and the current session. A low priming confidence can indicate that the user normally primes, but did not this time, or rarely primes, but did so this time:

if winning pattern contains at least one prime

$$primingConfidence = min ( 1 , \frac{p r i m e P r o b}{primingConfid e n ceWeigh t} )$$

else

$$primingConfidence = min ( 1 , \frac{1 - p r i m eProb}{primingConfide n ceWeigh t} )$$

4.3.4.5 Training Confidence

The algorithm refuses to label the first ignoreFirstSessions sessions, regardless of other confidence metrics, on the theory that the first handful of sessions are user training or otherwise not “normal.” This is achieved through a fifth, very simple confidence metric:

if sessionSerial>ignoreFirstSessions
trainingConfidence=1

else
trainingConfidence=0

where sessionSerial is this session's serial number, provided by the Segmentation module at Session object creation.

After all confidence metrics have been calculated, the overall session confidence is calculated as the minimum of the individual metrics. This overall confidence is compared against the configurable parameter confidenceThreshold to determine whether the session is labeled or unlabeled.

confidence=min(dataConfidence, expectedDoseConfidence, ambiguityConfidence, primingConfidence,trainingConfidence)

if confidence≥confidenceThreshold
session is labeled

else
session is not labeled

4.4 Session Analysis Algorithm Parameters

| | | Typical |
|---|---|---|
| Parameter | Description | value |
| maxInjectsSimple | How many injected dispenses allowed in a session for | 3 |
| | which the sum of all dispenses was less than the pen dialing | |
| | limit. The number of allowed injections is maxInjectsSimple + | |
| | floor(sum(dispenses)/dialMax). The IFU would suggest | |
| | maxInjectsSimple = 1, but it should be larger to | |
| | accommodate cartridge changes. | |
| minPrimes | How many flow-check dispenses (aka primes) to require before | 0 |
| | an injected dispense. It should be zero for normal algorithm | |
| | operation. Setting it to one would enforce IFU compliance. | |
| criticalDispenseInterval | For sessions with three or more dispenses, the time (in | 3.5 sec |
| | seconds) between consecutive dispenses beyond which the | |
| | second dispense is more likely to be an injection. | |
| dispenseIntervalFactor | Intra-session dispense intervals are classified as “short” or | 1.2 |
| | “long” by comparing them against criticalDispenseInterval. | |
| | The pattern weight is then multiplied by a factor | |
| | dispenseIntervalFactor \hat{ } (shortPP + longPI − shortPI − longPP) | |
| | where shortPP, longPI, etc. are the counts of long/short intervals | |
| | of the given type (prime followed by injection, prime | |
| | followed by prime, . . .) within that candidate pattern. | |
| primeProbFactor | Adjusts the pattern weighting for primeProb, the user's | 2.0 |
| | historical likelihood of priming. The weight factor is | |
| | primeProbFactor \hat{ } (2*primeProb − 1) | |
| | for patterns with priming, and | |
| | primeProbFactor \hat{ } (1-2*primeProb) | |
| | for patterns without. | |
| bolusPrimeDisparityFactor | For bolus drugs only, adjusts the pattern weighting for priming | 2.0 |
| | disparity. The disparity D is defined as the difference (in | |
| | units) between the largest and the smallest primes for the | |
| | session and the pattern being evaluated. The weight factor | |
| | is primeDisparityFactor \hat{ } (−D) | |
| bolusPrimeCrossoverSize | For bolus drugs, the dispense size which will receive a | 3.5 |
| | neutral weight of 1.0 when evaluated as a candidate prime. | |
| | Smaller dispenses are weighted >1 and larger dispenses | |
| | are weighted <1. | |
| bolusPrimeSizeSlope | For bolus drugs, the slope of the erf( ) S curve from which | 0.4 |
| | size-based weights are taken, when evaluating candidate | |
| | primes. | |
| bolusInjectSizeSlope | For bolus drugs, the slope of the erf( ) S curve from which | 0.2 |
| | size-based weights are taken, when evaluating candidate | |
| | injections. | |
| bolusInjectSizeOffset | For bolus drugs, an additional left/right shift applied to the | 1.0 |
| | erf( ) S curve from which size-based weights are taken, | |
| | when evaluating candidate injections. | |
| basalSplitDoseRatioMin | For basal drugs, when there are multiple candidate injections | 0.2 |
| | “x” in a pattern, this is the smallest ratio x/sum(all candidate | |
| | injections) which can be used for the setting the | |
| | adaptive crossover point. | |
| basalSizeSlopeMin | For basal drugs, the lower limit to the adaptive slope of the | 0.1 |
| | erf( ) S curve from which size-based weights are taken, for | |
| | both primes and injections. | |
| basalSizeSlopeMax | For basal drugs, the upper limit to the adaptive slope of the | 50.0 |
| | erf( ) S curve from which size-based weights are taken, for | |
| | both primes and injections. | |
| dataConfidenceWeight | From 0 to 1, adjusts the influence of the “data score” on the | 0.5 |
| | data confidence. | |
| expDoseConfidenceWeight | From 0 to 1, adjusts the influence of the discrepancy between | 0.15 |
| | the expected dose and the estimated dose (in standard | |
| | deviations) on the expected-dose confidence. A setting | |
| | of 0.15 results in an expected-dose confidence of 0.7 when | |
| | the discrepancy is two standard deviations. | |
| ambigConfidenceWeight | From 0 to 1, adjusts the influence of the dose posterior | 0.7 |
| | distribution “ambiguity” on the ambiguity confidence. Ambiguity | |
| | is the reciprocal of the ratio of the peak in the posterior | |
| | distribution (corresponding to the estimated dose) to the sum | |
| | of the remainder of the distribution. A setting of one results | |
| | in an ambiguity confidence of zero when the peak of the | |
| | posterior is 0.5 (implying that the remainder of the posterior | |
| | distribution sums to 0.5). | |
| primingConfidenceWeight | From 0 to 1, adjusts the influence of the user's priming | 0.5 |
| | consistency on the priming confidence. A setting of 0.5 causes | |
| | the priming confidence to be one as long as the user's | |
| | historical priming probability (primeProb) is >0.5 (if they | |
| | primed this time), or <0.5 (if they did not prime this time). | |
| confidenceThreshold | From 0 to 1, controls the session labelling threshold. The | 0.7 |
| | overall confidence is the minimum of all of the separate | |
| | confidence values. The session is labelled if and only if this | |
| | overall confidence >= confidenceThreshold. | |
| ignoreFirstSessions | A user's first ignoreFirstSessions sessions are always unlabelled, | 4 |
| | regardless of the other confidence measures, to avoid confusing | |
| | the algorithm during the pen demonstration/user education period. | |

### 5. Definitions & Terminology

This list contains definitions of abbreviations and terms used in this document.

| Term | Definition |
|---|---|
| Dispense | A single mechanical activation of a drug-delivery device. A dispense may be a |
| | flow check or prime, an injection, air shot or wet shot, etc. The input data for |
| | the algorithm are a series of time-stamped dispense records. |
| Dose | The quantity of insulin (or other drug) that is actually injected into the body |
| | during a session. In some cases it may be split across more than one injected |
| | dispense. |
| Estimated | The estimated dose is the main output of the algorithm - its best guess of the |
| Dose | true session dose based on all available information, including the details of |
| | the current session. |
| Expected Dose | For each session, an expected dose may be determined based on the user's |
| | dosing history. The expected dose does not take into account the actual |
| | dispenses comprising the session, it is merely an expectation based on the |
| | past. |
| Flow-checks | Dispenses that are not entering the body - e.g. priming a pen before use |
| Prime | For brevity prime is used in code and refers to flow checks |
| Priming | The act of performing a flow check. |
| Injections | Dispense that is assumed to have entered the body. |
| Label | A formal claim about the session dose. The algorithm always outputs an |
| | estimated dose, but it may choose not to label the session if confidence is not |
| | sufficiently high (how high depends on the tuneable algorithm parameters). |
| | When a session is labelled, this tells the client app that the true session dose is |
| | known with high confidence. Note that this is fully equivalent to labelling each |
| | dispense as either an injection or a flow check. |
| Session | A group of dispenses which are clustered in time, interpreted as the user |
| | taking a single dose of the drug. A session often consists of one or more |
| | flow-check, followed by a single injected dispense, but other patterns are of |
| | course possible. The key idea is that the session, however complex, is associated |
| | with a single dose. |
| Training | The first numbers of sessions are not labelled. This ensures the algorithm has |
| | gathered enough data on the user to perform its predictions. |

Having illustrated aspects of the present invention in a first example and having exemplified aspects of the invention by a detailed description of specific algorithm components, next a second example will be described in which reference is made to the aspects of the disclosed algorithm components.

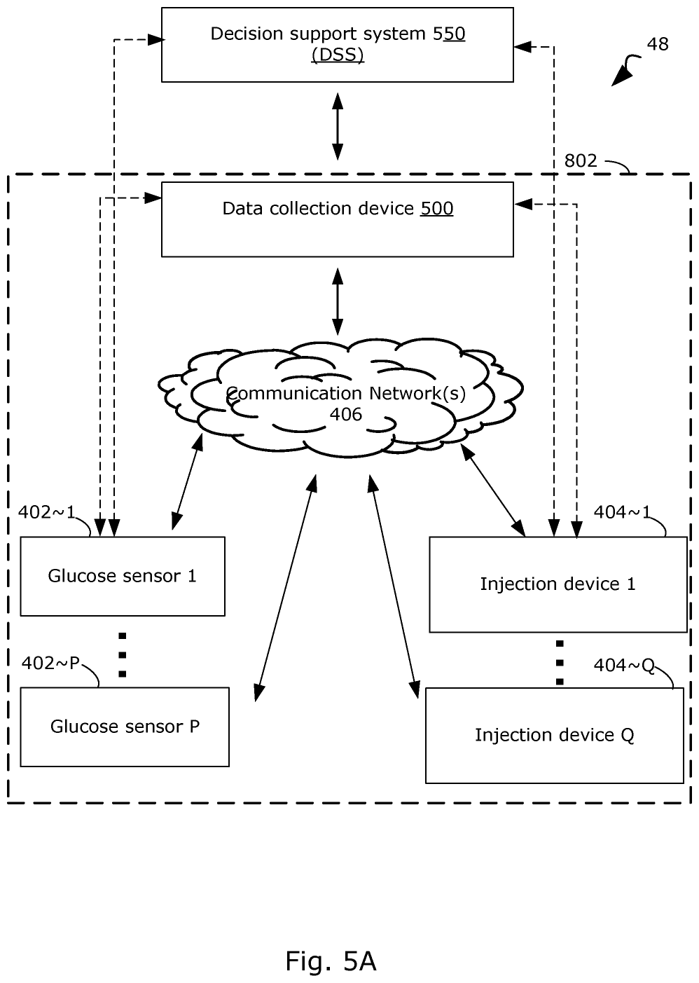

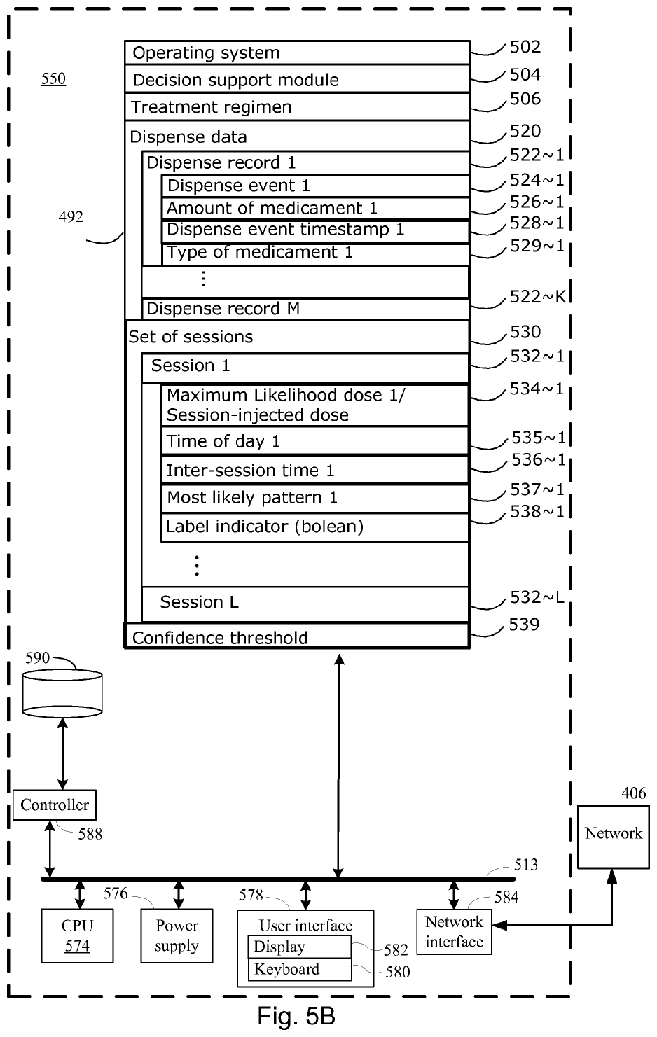

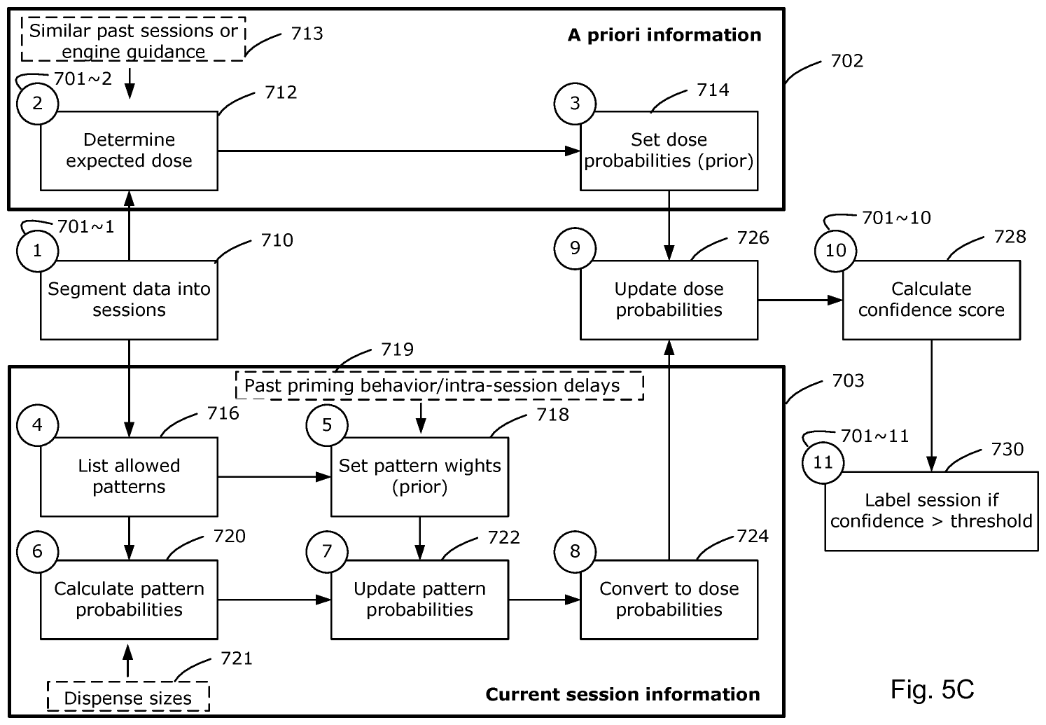

FIG. 5A illustrates an example of an integrated medical system 802 for collection of dispense data from one or more injection devices 404. The illustrated embodiment also shows that system optionally also can be adapted to collect blood glucose data from one or more glucose sensors 402. The medical system 802 also includes a processor although it is not illustrated on FIG. 5A.

With the integrated system 802, data from the one or more connected injection devices 404, used to apply a treatment regimen to the subject, is obtained as a set of medicament dispense records 522 in a plurality of dispense data 520 or a dispense data set 520. Each dispense record comprises a timestamped event specifying an amount of dispensed blood glucose regulating medicament that the subject received as part of the treatment regimen. The time stamped event specifying the amount of blood glucose regulating medicament is automatically obtained in the sense, that the subject or user of the injection device is not required to perform an active step in order to obtain an electronic or digital time stamp and/or an electronic or digital amount of blood glucose regulating medicament. These data are automatically generated by the injection device upon application of injection, i.e., the injection is applied by the subject or user in order to expel an amount of medicament, but the generation of data is provided irrespective of the user's intention, when he or she uses the device. Also, in some embodiments, autonomous timestamped glucose measurements of the subject are obtained. In such embodiments, the autonomous glucose measurements are filtered and stored in nontransitory memory. The plurality of dispense records of the subject taken over a time course are used to provide input to a decision support system (DSS) 550 adapted to enhance the quality of the raw data stream and convert it into a data structure, which reliably enables the prediction of injected medicament.

A detailed description of a medical system 48, for collecting raw dispense data from one or more injection devices, and enhance the quality of the raw data stream and convert it into a data structure, which reliably enables the prediction of injected medicament, is described in conjunction with FIGS. 5A and 5B. As such, FIGS. 5A and 5B collectively illustrate the topology of the system in accordance with the present disclosure. In the topology, there is a decision support system 550 for enhancing the data quality of dispense data, in order to be able provide reliable decision support to a subject following a treatment regimen 506, a device for data collection (“data collection device 500”), one or more injection devices 404 for injecting medicaments into the subject, and optionally one or more glucose sensors 402 associated with the subject. Throughout the present disclosure, the data collection device 500 and the decision support system 550 will be referenced as separate devices solely for purposes of clarity. That is, the disclosed functionality of the data collection device 500 and the disclosed functionality of the dose history communication device 550 are contained in separate devices as illustrated in FIG. 5A. However, it will be appreciated that, in fact, in some embodiments, the disclosed functionality of the data collection device 500 and the disclosed functionality of the decision support system 550 are contained in a single device. In some embodiments, the disclosed functionality of the decision support system is contained in a smart phone or a cloud service. In some embodiments the data quality enhancing functionality may be in a separate device, e.g. a quality enhancing device, which is different from the device comprising decision support system 550. The data quality enhancing device can then be in communication with the decision support device comprising the decision support system 550. In some embodiment the data collection device is an add-on device 300 as illustrated on FIGS. 1C and 1D, and in other embodiments the data collection device is an integrated device of the one or more injection devices 404.

Referring to FIG. 5B, in some embodiments, the treatment regimen 506 comprises a bolus insulin medicament dosage regimen with a short acting insulin medicament or a basal insulin medicament dosage regimen with a long acting insulin medicament. In some embodiment the treatment regimen may also comprise a dosage regimen with a medicament comprising a GLP-1 receptor agonist as for example liraglutide or semaglutide.

Referring to FIG. 5A, the decision support system 550 enhances the data quality of dispense data, in order to be able provide reliable decision support to a subject following the treatment regimen 506. To do this, the data collection device 500, which is in electrical communication with the decision support system 550, receives a plurality of blood glucose regulating medicament dispense records over a time course, each dispense record 522 comprising (i) a blood glucose regulating medicament dispense event 524 including an amount of insulin medicament 526 dispensed by the subject using a respective injection device 404 in the one or more injection devices, and (ii) a corresponding electronic dispense event timestamp 528 that is generated by the respective injection device upon occurrence of the blood glucose regulating medicament injection event. If more than one medicament is applied, (iii) a respective type of blood glucose regulating medicament 529, dispensed by the subject from one of short and long acting insulin medicament. In some embodiments, the data collection device 500 also receives glucose measurements from one or more glucose sensors (e.g., continuous glucose monitors/sensors) 502 used by the subject to measure glucose levels. In some embodiments, the data collection device 500 receives such data directly from the injection devices 404 and/or glucose sensor(s) 502 used by the subject. For instance, in some embodiments, the data collection device 400 receives this data wirelessly through radio-frequency signals. In some embodiments, such signals are in accordance with an 802.11 (WiFi), Bluetooth, or ZigBee standard. In some embodiments, the data collection device 200 receives such data directly, analyses the data, and passes the analysed data to the dose history communication device 250. In some embodiments, an injection device 404, which can be an insulin pen, and/or a glucose sensor 402 includes an RFID tag and communicates to the data collection device 500 and/or the decision support system 550 using RFID communication.

In some embodiments, the data collection device 500 and/or the decision support system is not proximate to the subject and/or does not have wireless capabilities or such wireless capabilities are not used for the purpose of acquiring medicament dispense data, autonomous glucose data, and/or life-style related measurement data. In such embodiments, a communication network 406 may be used to communicate insulin medicament dispense data from the one or more injection devices 404 to the data collection device 500 and/or the decision support system, and/or autonomous glucose measurements from the glucose sensor 402 to the data collection device 500 and/or the decision support system 550.

Examples of networks 406 include, but are not limited to, the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (Vol P), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of the present disclosure.

In some embodiments, the data collection device 500 and/or the decision support system 550 is part of an insulin pen. That is, in some embodiments, the data collection device 500 and/or the decision support system 550 and an injection device 404 are a single device.

Of course, other topologies of the system 48 are possible. For instance, rather than relying on a communications network 106, the one or more injection devices 404 and the optional one or more glucose sensors 402 may wirelessly transmit information directly to the data collection device 500 and/or decision support system. Further, the data collection device 500 and/or decision support system may constitute a portable electronic device, a server computer, or in fact constitute several computers that are linked together in a network or be a virtual machine in a cloud computing context. As such, the exemplary topology shown in FIG. 1 merely serves to describe the features of an embodiment of the present disclosure in a manner that will be readily understood to one of skill in the art.

Referring to FIG. 5B, in typical embodiments, the decision support system 550 comprises one or more computers. For purposes of illustration in FIG. 5B, the decision support system 550 is represented as a single computer that includes all of the functionality for enhancing the data quality of raw dispense data, in order to be able provide reliable decision support to a subject following the treatment regimen 506. However, the disclosure is not so limited. In some embodiments, the functionality for enhancing the data quality of dispense data is spread across any number of networked computers and/or resides on each of several networked computers and/or is hosted on one or more virtual machines at a remote location accessible across the communications network 406. One of skill in the art will appreciate that any of a wide array of different computer topologies are used for the application and all such topologies are within the scope of the present disclosure.

Turning to FIG. 5B with the foregoing in mind, an exemplary decision support system 550 for enhancing the data quality of raw dispense data comprises one or more processing units (CPU's) 574, a network or other communications interface 584, a memory 492 (e.g., random access memory), one or more magnetic disk storage and/or persistent devices 590 optionally accessed by one or more controllers 588, one or more communication busses 513 for interconnecting the aforementioned components, a user interface 578, the user interface 578 including a display 582 and input 580 (e.g., keyboard, keypad, touch screen), and a power supply 576 for powering the aforementioned components. In some embodiments, data in memory 492 is seamlessly shared with non-volatile memory 590 using known computing techniques such as caching. In some embodiments, memory 492 and/or memory 590 includes mass storage that is remotely located with respect to the central processing unit(s) 574. In other words, some data stored in memory 492 and/or memory 590 may in fact be hosted on computers that are external to the decision support system 550, but that can be electronically accessed by the decision support system 550 over an Internet, intranet, or other form of network or electronic cable (illustrated as element 406 in FIG. 3) using network interface 584.

In some embodiments, the memory 492 of the decision support system 550 for enhancing the data quality of raw dispense data from a data collection device 500 stores:
an operating system 502 that includes procedures for handling various basic system services,
a decision support module 504,
a treatment regimen 206 which the subject is engaged in,
a dispense data set 520 automatically obtained from one or more injection devices used by the subject to apply the treatment regimen, the dispense data set comprising a set of dispense records over a time course, each respective medicament dispense record 522 in the set of medicament records compress: (i) a respective medicament dispense event 524 including an amount of medicament 526 dispensed by the subject using a respective injection device 104 in the one or more injection devices, (ii) a corresponding electronic dispense event timestamp 228 within the time course that is automatically generated by the respective injection device 104 upon occurrence of the respective medicament injection event, (iii) a type of medicament 529, if more than one type of medicament is dispensed,
a set of dispense sessions 530 within the time course, wherein
each respective session 523 comprises: (i) a Maximum Likelihood dose 534 indicating the session-injected dose, (ii) a time of day 536 registration indicating the time of the day the session occurred, (iii) inter-session time 537 registration indicating the time since last session, (iv) a most likely dispense pattern 537, and (v) a label indicator 538 which is a Boolean indicating whether or not the most likely pattern the session can be labelled,
a confidence threshold 539 determining the binary value of the label indicator.

In some embodiments, the decision support module 504 is accessible within any browser (phone, tablet, laptop/desktop). In some embodiments the decision support module 504 runs on native device frameworks, and is available for download onto the device comprising the decision support system 550 running an operating system 502 such as Android or iOS.

In some implementations, one or more of the above identified data elements or modules of the decision support system 550 for enhancing data quality of raw dispense data are stored in one or more of the previously described memory devices, and correspond to a set of instructions for performing a function described above. The above-identified data, modules or programs (e.g., sets of instructions) need not be implemented as separate software programs, procedures or modules, and thus various subsets of these modules may be combined or otherwise re-arranged in various implementations. In some implementations, the memory 492 and/or 590 optionally stores a subset of the modules and data structures identified above. Furthermore, in some embodiments, the memory 492 and/or 590 stores additional modules and data structures not described above.

In some embodiments, a decision support system 550 for enhancing the data quality of raw dispense data is a smart phone (e.g., an iPhone), laptop, tablet computer, desktop computer, or other form of electronic device (e.g., a gaming console). In some embodiments, the decision support system 550 is not mobile, and in some embodiments it is.

FIG. 5C illustrates a method according the present disclosure of enhancing the quality of dispense data from the data collection device 200, and for the purpose of describing the method the following terminology is used.

A dispense or dispense event is a pen activation, whether or not insulin comes out of the needle or is injected into the body.

A prime or priming event is any dispense preparatory to an injection. This includes priming a new cartridge, but also routine flow checks before each injection.

An injection or injection event is a dispense event, wherein the medicament is presumed injected into the body.

A session is a sequence of “prime” and “injection” dispenses, clustered in time, during which the user intends to take a single target dose of insulin. A single session may have multiple injections because of dose splitting, dial limitation or a cartridge change.

A pattern is one particular sequence of primes and injections, often written in shorthand like “ppi” (prime, prime and injection) or “pii” (prime, injection and injection). Each pattern is an interpretation of the dispenses comprising a session. Because we know the amount of medicament 526 of each dispense, identifying the correct pattern is equivalent to determining the session-injected dose 534.

A session-injected dose is how much insulin the user intended to inject during the session.

A Maximum-likelihood dose is the rule-based algorithm's best estimate of the session-injected dose.

A Labeling rate is the fraction of sessions for which a Maximum Likelihood dose is communicated back to the decision support system to assign the session injected dose with the value of the Maximum Likelihood dose, and to label the session with an estimated pattern. The user will be asked to manually label the rest. The decision to label a session is based on a confidence score. The confidence score is to be evaluated against a confidence threshold, which affects the labeling rate. The labeling rate can be set anywhere from 0% to 100% by choice of algorithm parameters, i.e., the choice of confidence threshold.

With reference to FIG. 5C, reference number 701 indicates an index number for numbering the individual steps in the process. The index number 701˜1=1 for process step 710, and 701˜2=2 for process step 712 and so forth. The rectangle 702 indicates processes relating to determining a prior probability distribution of a dose based on estimates of previous sessions. The rectangle 703 indicates processes relating to determining the probability of dose based on information of the current session.

Segment Data into Sessions

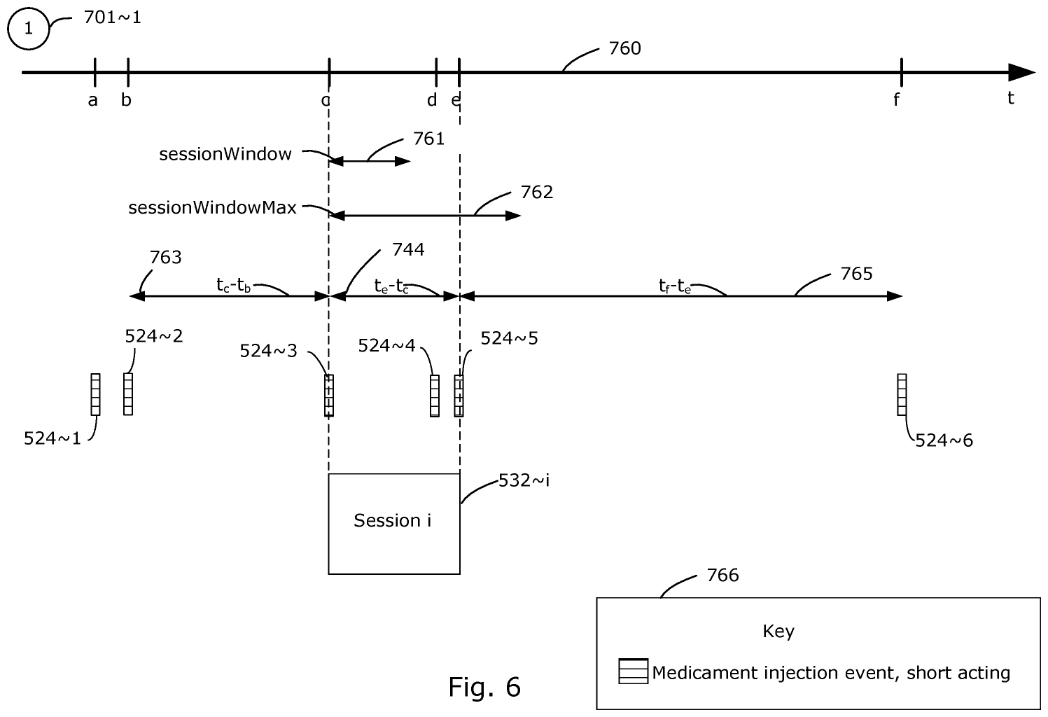

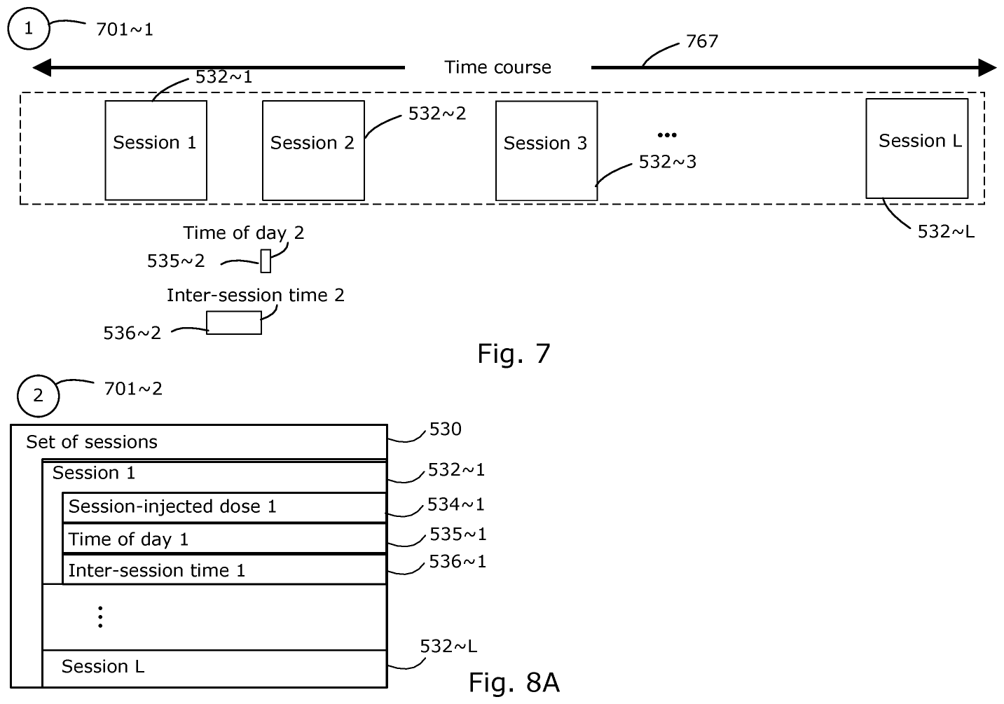

Block 710. The solution of connecting an injection device provides a stream of time-stamped dispense records. These must be segmented into logical sessions before the dose estimation can proceed, as indicated in step 710 in FIG. 5C and further illustrated in FIGS. 6 and 7. A session corresponds to the user deciding to take some insulin and completing that task.

Segmentation is controlled by three parameters. The initial dispense starts a new session and zeros a timer, and the next dispenses are automatically included in this session until sessionWindow 761 seconds have elapsed, as illustrated in FIG. 6. Later dispenses may still be included, provided the ratio between the resulting session length and the gap on either side is less than sessionLengthRatio, and the timer is still less than sessionWindowMax. Once this is no longer true, the next dispense starts a new session and the process repeats.

In the example below, and as illustrated in FIG. 6, with dispenses a, b, c, d, e, f, {a, b} comprise one session, because t_{b}−tₐ<sessionWindow (761). Dispense event c starts a new session, because t_{c}−tₐ>sessionWindowMax (762). Dispense events {c, d, e} comprise a session as:
sessionWindow (761)<tₑ-t_{c}<sessionWindowMax (762), provided that
(tₑ−t_{c})/(t_{f}−tₑ)<session Length Ratio, and
(tₑ−t_{c})/(t_{c}−t_{b})<sessionLengthRatio.

Dispense f starts a new session, because t_{f}−t_{c}>sessionWindowMax (762).

Determine Expected Dose

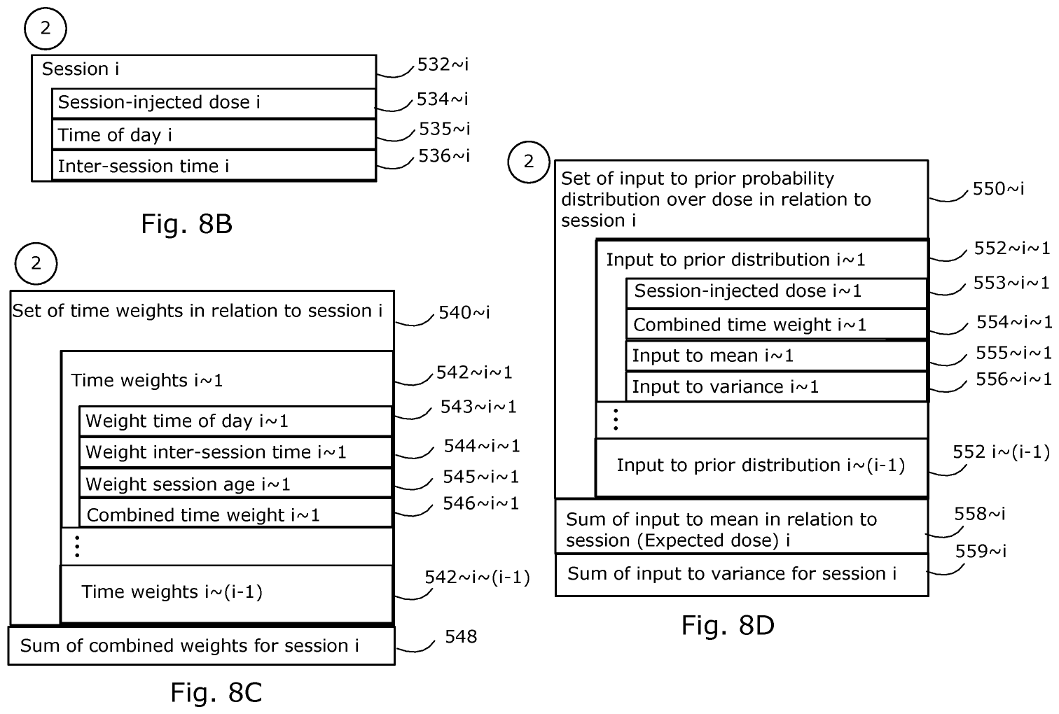

Block 712. The step of determining the expected dose is conducted in step 712, and further illustrated in FIGS. 8A to 8D. Even without any data on the current session, it is possible to determine that some doses are more likely than others. Information on likelihood of a dose can be can be obtained by dose guidance given by the decision support system or similar past or prior sessions 713. Expected dose based on history of prior sessions is selected by setting expectedDoseMethod=‘history’ and adjusting the doseHistory parameters. Similarly if setting expectedDoseMethod=‘dose guidance’ dose guidance is used. The doseHistory parameters control a weighted average with weights set by similarity in time of day, similarity in time since last session and age of data, with recent doses weighted more strongly. The decrease of the weighing function can be Gaussian, exponential or linear depending on the nature of the data. FIG. 8A illustrates the construction of a data structure comprising the set session created in the sectioning step 710. The set of session 530 comprises a number of sessions L, and each session comprises a session injected dose 534, which is estimated by the described algorithm, a time of day 535, and an inter-session time 536.

It is worth noting that all three of these weight contributors are completely adjustable in the code and needs to be adjusted to the application. The exponential decay rate for discounting the weight over time can be set with a half-life of 28 days in the bolus regimen and 10 days in the basal regimen. For similarity in time it is e.g. possible to use a Gaussian decay factor, wherein the weight is 50% at +/−3 hours. In the case of inter-session time length the Gaussian decay factor can for example be 50% at +/−2.5 hours in the bolus regimen and 1e20 for bolus, which effectively disables this inter-session time weight in the basal regimen. For the application of basal insulin the injections should be more regular.

Therefore, for each prior session record 532 is associated a session-injected dose 524, a time of day 535 and an inter-session time specifying the time to the previous session, as illustrated in FIG. 8A. For each session i within the set running from 1 to L and which session is illustrated as a data structure in FIG. 8B, is calculated a corresponding weight time of day 543, a weight inter-session time 544 and weight session age 545, which is illustrated as a corresponding data structure in FIG. 8C. The three weights 543, 543, 545 can be combined to a combined time weight 546. The weights are evaluated from a time in the past and until the session just before (i−1) the current session i.

The value of the sum of combined time weights for prior sessions 548 can be used to determine whether or not there is enough data to continue to determine the prior probability distribution. The value of the sum 548 is compared to an empirically estimated threshold.

If there is enough similar prior data, the combined weight can be multiplied with the session injected dose for each session to provide the contribution to the mean of the distribution, which is referred to as the input to mean 555. By adding all the inputs to mean 555 from each session prior to the current session the weighted mean for the prior probability distribution is obtained, which also can be referred to as the expected dose 558. In the same way the weighted variance can be calculated by calculating an input to the variance (wᵢ²σᵢ²) 556 and adding all the inputs from the session prior to the current session. wᵢ denotes the weight and σᵢ² denotes the variance.

FIG. 8D illustrates the current session that the current session i is associated with set of input to prior probability 550, and each input to prior distribution 552 comprises a session-injected dose 553. The session-injected dose 553 is numerically the same as the session-injected dose 534, however it is given a new reference number to illustrate that it here is used for calculating the prior distribution. The input to prior distribution also comprises the combined time weight 554, the input to mean 555, and the input to variance. The inputs 555, 556 are summed to provide the sum of input to mean in relation to session i (558), and the sum of input to variance for session 559. The session-injected dose 553 is numerically the same as the session-injected dose 534, however it is given a new reference number to illustrate that it here is used for calculating the prior distribution. Similar, the combined time-weight 554 is numerically the same as the combined time weight 546, however it is given a new reference number to illustrate that it here is used for calculating the prior distribution.

Set Dose Probabilities

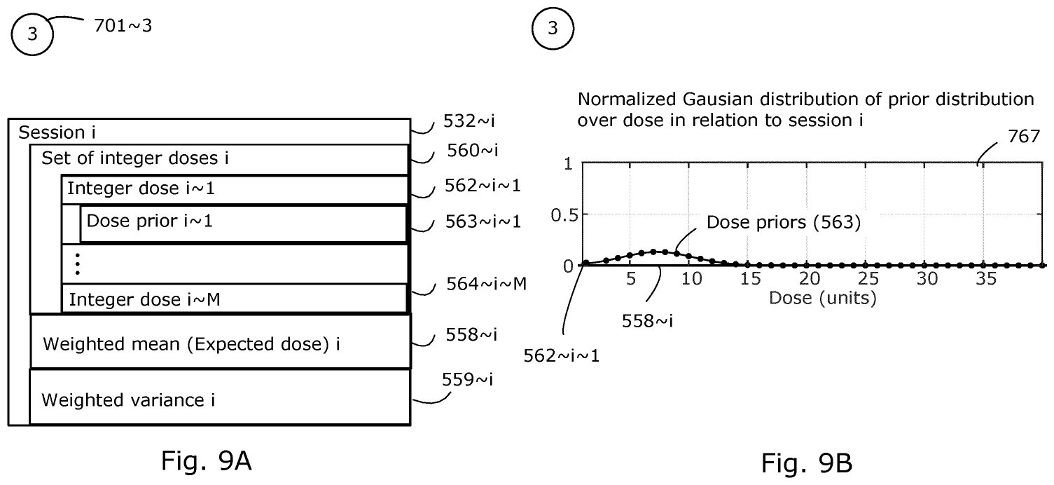

Block 714. The weighted mean 558 and the weighted variance 559 are used to calculate the prior dose probabilities for integer doses as illustrated with the data structure illustrated in FIG. 9A and the Gaussian distribution shown in FIG. 9B. This step is indicated with box 714 in FIG. 5. The prior distribution may be uniform, i.e., constant, if there is not enough prior knowledge or dose guidance. This is common when there is no decision support and the user is too new in the system to have accumulated a meaningful dosing history.

FIG. 9A shows an example of a data structure that can be used in step 714, wherein a set of integer doses 560 are created to evaluate the dose probabilities, as the session injected dose is assumed to be an integer. If the session-dose is a real number including fractions of doses, as set of real does corresponding to possible session-doses should be created. The possible session-doses is determined by the nature of the injection device. In this example, for each integer dose 562 is evaluated a dose prior 563. The dose prior is evaluated based on the prior distribution with mean and variance corresponding to the expected dose 558, and the weighted variance 559. The expected dose 558 is also indicated in FIG. 9B along with the dose priors 563.

Hereafter, the method proceeds to evaluating information based on the current session, which is indicated in step 716, in FIG. 5.

List of Allowed Patterns

Block 716. A session with N dispenses can be interpreted in up to 2^{N} ways, e.g. with N=2 the number of patterns is 4 and with the notation where “p” indicates a prime and “i” indicates an injection the patterns can be either “pp”, “pi”, “ip”, or “ii”. But not all of these are plausible, and for the described example it is assumed that (1) every session results in an injection, and (2) primes don't happen after injections unless the session still has another injection coming. Primes after injections can happen in a session if the cartridge is changed in a durable pen, or if a new prefilled pen is used.

Cartridge changes are a particular challenge on reusable pens where there is no automatic detection mechanism. The current algorithm assumes we are always notified about cartridge changes or when a new pen is used. This can be achieved, e.g., by tracking usage of the current cartridge and, once it nears end-of-life, “nagging” the user through the UI to confirm/deny if it has been replaced. If a new pen is used, the new pen will identify itself with a unique identification code.

The number of allowable injections also depends on the likelihood of a split dose, and the configurable parameter maxInjectsSimple. Thus by excluding a cartridge change and with setting maxInjectsSimple=2 the allowable patterns for up till 4 dispenses are as listed in the below table.

| N | Allowable patterns |
|---|---|
| 1 | “I” |
| 2 | “pi”, “ii” |
| 3 | “ppi”, “pii” |
| 4 | “pppi”, “ppii” |

The table can be extended to any number of dispenses. In this example, the number of primes is not capped but the number of injections is. If the expected dose were larger than the pen's dialling limit, another injection would be allowed, permitting “iii” when N=3 and “piii” when N=4. If the session contained a cartridge change, the patterns “ipi”, “ippi”, “pipi” would become possible too.

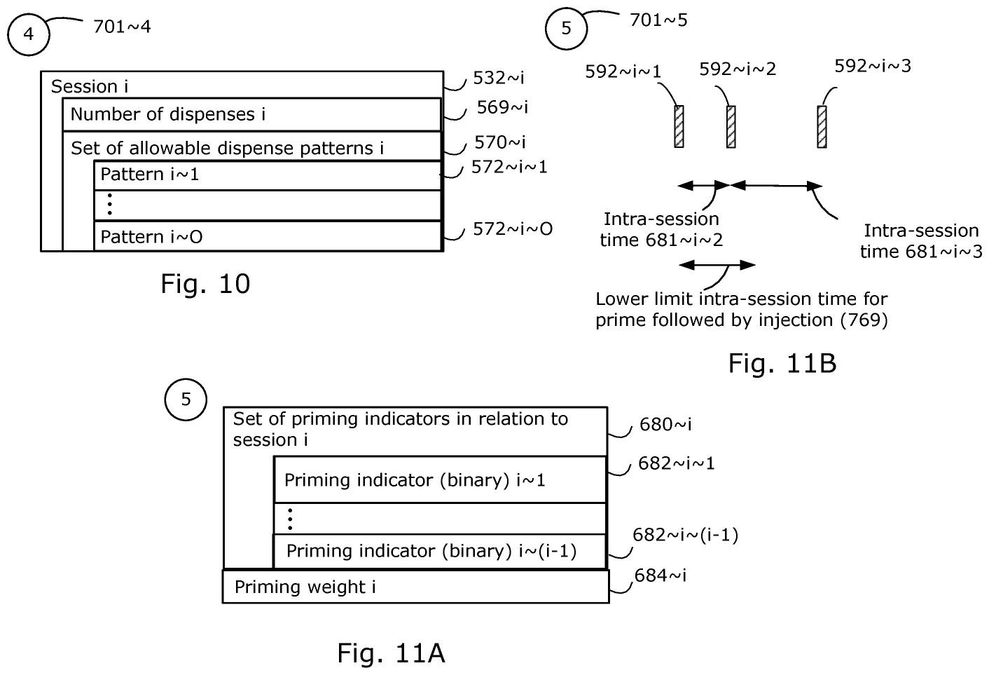

FIG. 10 shows an example of a possible data structure for use in the method, wherein the data structure illustrates a structuring of the number of dispenses 569 for session I, and the set of allowable dispense patterns 570 comprising O patterns 572.

Set Pattern Weights (Pattern Prior)

Block 718. Without looking at the dispense sizes, some patterns are more likely than others. This is analogous to step 714, where the prior probability was found for the possible session-doses. The inputs to the prior distribution over patterns are (i) the user's past priming behaviour, if they have not primed in the past they are unlikely to start doing so now, and vice versa. (ii) Time intervals between dispenses in the current session. Specifically, intervals longer than 3.5 seconds are more likely to precede an injection, while intervals shorter than 3.5 seconds are more likely to precede a prime. However, the determining interval could also be close to 3.5 seconds as for example 3 and 4 seconds.

FIG. 11A illustrates a data structure for a session i comprising a set of priming indicators 680 comprising a priming indicator 682 for all the previous sessions. Each priming indicator 682 is a binary, e.g., 1 or 0, and can be used to calculate a priming weight 684 for the current session, which then is the fraction of sessions with primes. Other linear or exponential weight functions using the priming indicator as argument can be contemplated.

FIG. 11B illustrates the sectioned dispense events 592 in session i. There are 3 dispense events 592 in the section which results in 2 intra-session times 681, as the intra-session time 681 defines the time before the dispense event in consideration. Therefore, the first dispense event 592˜i˜1 cannot have an intra-session time 681. The lower limit intra-session time 769 indicates a parameter determining a preference for either a prime or an injection. The lower limit intra-session time 769 can for example be 3.5 s. In the illustrated example the intra-session time 681˜i˜2 is smaller than the lower limit intra-session time 769, which means that the dispense event 592˜i˜2 is likely a priming event. The dispense event 592˜i˜2 is most likely an injection, as it is larger than the lower limit intra-session time 769.

After it has been decided which patterns are allowable, each one has equal probability to begin with. These probabilities are adjusted based on two factors, in the described embodiment: (1) the user's “priming probability” (based on how often we have observed them performing flow-checks in the past), and (2) the timing between dispenses within this session (intra-session timing).

For item (1), there is maintained a “priming probability” between 0 and 1. The priming weight can be understood as the fraction of past sessions in which the user performed at least one flow-check or priming dispense. In another embodiment is applied an exponential “forgetting factor” so that long-ago sessions do not count as much as more recent sessions, otherwise, if a user changed their behaviour, the algorithm would be too slow to adapt. The way this affects the pattern weights is as follows: Initially each weight is 1.0. For each pattern with priming, the weight is modified as:

patternWeight=patternWeight*2^{(2*primeProb-1)}

and for each pattern without priming, we modify the weight as

patternWeight=patternWeight*2^{(1-2*primeProb)}

It is noticed that if the user exhibits no tendency either way (primeProb=0.5), then the weights are multiplied by one and remain the same. If the user tends to prime, the patterns with priming get a larger weight and the patterns without priming get a smaller weight, and vice versa, if the user tends not to prime.

For item (2), investigations show that a 3.5-second gap between dispenses is a good cut-off, longer gaps are more often associated with “pi” (flow check followed by injection), whereas shorter gaps are more often associated with “pp” (two flow checks). Note that this has no effect at all if there are only two dispenses in the session, because the pattern “pp” is not allowed anyway. There has to be at least one injection. But for sessions with three or more dispenses, the information on intra-session time can be used.

In an exemplary embodiment the weight calculation can be calculated in the following way:

patternWeight=patternWeight*1.2^{(length(shortPPs)+length(longPIs)})/1.2^{(length(longPPs)+length(shortPIs))},

where “length(longPPs)” is a count of the number of “pp” dispense pairs, “length(longPIs)” is a count of the number of “pi” dispense pairs in the pattern, where the actual time intervals were either long, e.g., t>3.5 s. “length(shortPPs)” is a count of the number of “pp” dispense pairs, “length(shortPIs)” is a count of the number of “pi” dispense pairs in the pattern, where the actual time intervals were short, e.g., t<3.5 s, in this example.

Therefore, for example if a current session of 3 dispenses, with 2 seconds between dispense 1 and 2, and 5 seconds between dispense 2 and 3 is considered. The allowed patterns are: ppi, pii.

For the pattern ppi the dispense pairs are counted as:

length(longPPs)=0,length(shortPIs)=0 and (length(longPPs)+length(shortPIs))=0, and

length(shortPPs)=1,length(longPIs)=1 and (length(shortPPs)+length(longPIs))=2.

The corresponding pattern weights can then be calculated as:

patternWeight(“ppi”)=patternWeight(“ppi”)*1.2²/1.2¹=patternWeight(“ppi”)*1.2²

For the pattern pii the pattern pairs are counted as:

length(longPPs)=0,length(shortPIs)=1 and (length(longPPs)+length(shortPIs))=1, and

length(shortPPs)=0,length(longPIs)=1 and (length(shortPPs)+length(longPIs))=1.

The corresponding pattern weights can then be calculated as:

patternWeight(“pii”)=patternWeight(“pii”)*1.2¹/1.2¹=patternWeight,

whereby patternWeight(“ppi”)>patternWeight(“pii”), and the pattern prior will in thereby in this example increase the probability of labelling the session as “ppi”, when we the pattern probabilities is multiplied with the pattern weights and normalized to 1, as will be described in relation to block 722.

Calculate Pattern Probabilities

Block 720. For each dispense in the session is used a probability curve to express the probability of a dispense being an injection or a prime based on dose size, i.e., a P(injection)-versus-dispensesize curve or a P(prime)-versus-dispense-size curve. The larger the dispense size, the more likely it is to be an injection and the less likely it is to be a prime. The dispense sizes of the dispenses in each session is input 721 to the calculation of pattern probabilities.

The actual curve used can for example be erf(x) (integral of a Gaussian) which has a nice S shape. The scaling depends on drug type: For basal insulins is used the tracked average prime/inject sizes for the current user, this makes the algorithm less likely to reject larger priming dispenses (3-4 units or even higher), when it is known that the injection is usually much larger, e.g. 20 units. For bolus insulins, injected doses are less consistent and often smaller, so in this example the erf(x) is adopted to be a fixed S curve with the 50/50 crossover between prime/inject at 4 units. Additionally, if all dispenses in the session are <=4 units we automatically assume that candidate injections are, in fact, injections. This avoids rejecting very small doses. Once the erf(x) curves are determined, the probability of each pattern becomes a simple product of P(injection) and P(prime)=1-P(injection) factors. E.g. in a session with dispenses {3, 8} the probability of pattern “pi” is [1−P(3 is an injection)] *P(8 is an injection).

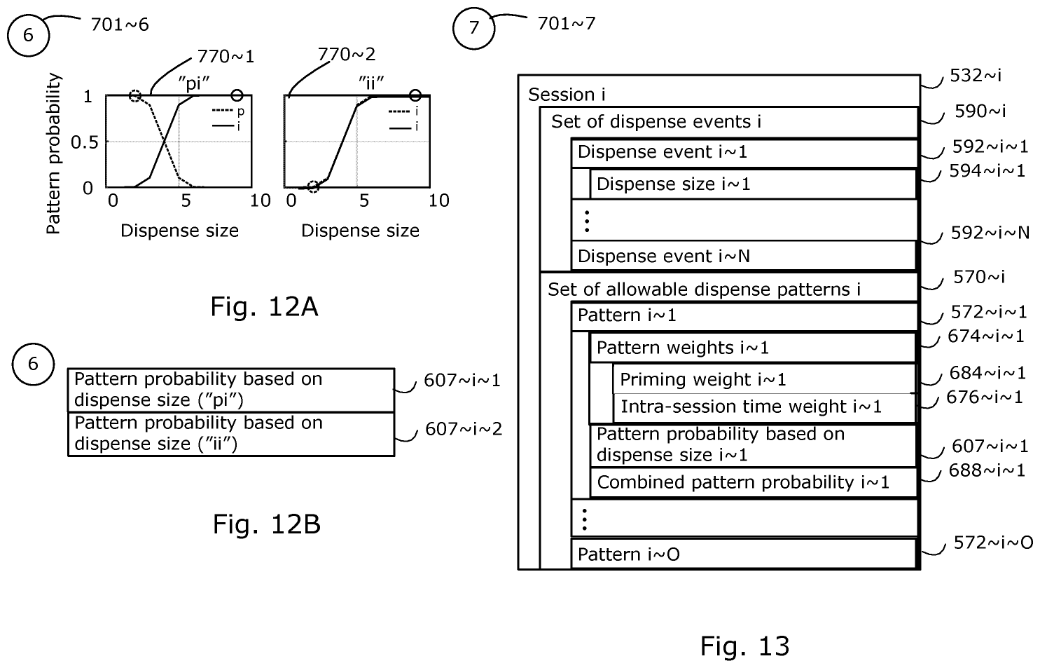

FIG. 12A illustrates the probability curve erf(x) for 2 bolus dispenses. In the left panel 770-1 is shown the probability for the pattern “pi”. The dotted curve shows the probability for the dispense being a prime, and if the dispense size is below 4 units the dispense event is most likely a prime. Similarly, the solid curve shows the probability of the dispense being an injection as a function of dispense size. The right panel 770-2 shows the probability for the pattern ii. The circles indicate actual dispense sizes for the current session. FIG. 12B illustrates a data structure for the pattern probabilities 607, which are associated with the current session i.

Update Pattern Probabilities (Using Prior)

Block 722. After the size-based probabilities are obtained for each allowable pattern, Bayes' theorem is applied to factor in the pattern weights from step 718. The size-based probabilities are multiplied by the pattern weights (prior distribution) and the result is normalized so the overall pattern probabilities sum to 1.

FIG. 13 show a data structure illustrating the structuring of the sectioned dispense events 592 and the allowable patterns 572 for the current session i. The allowable patterns are associated with the pattern weights 674 comprising the priming weight 684 and the intra-session time weight 676, and the pattern probability based on dispense size 607. A combined pattern probability 688 can be calculated based on patterns weights 674 and pattern probability based on dispense size 607, as described above.

Convert Pattern Probabilities to Dose Probabilities

Block 724. The mapping from pattern to dose is “many to one.” That is, multiple patterns might result in the same session dose but there is no ambiguity going from pattern to dose.

Example: Session {1, 2, 1, 7}. Allowed patterns are “pppi”, “ppii”, “pipi”, “ippi”.

The possible doses are:
7 units (“pppi”)
8 units (“ppii”, “ippi”)
9 units (“pipi”)

Therefore,
P(dose=7)=P(pattern is “pppi”)
P(dose=8)=P(pattern is “ppii”)+P(pattern is “ippi”)
P(dose=9)=P(pattern is “pipi”)
P(dose<7)=P(dose>9)=0

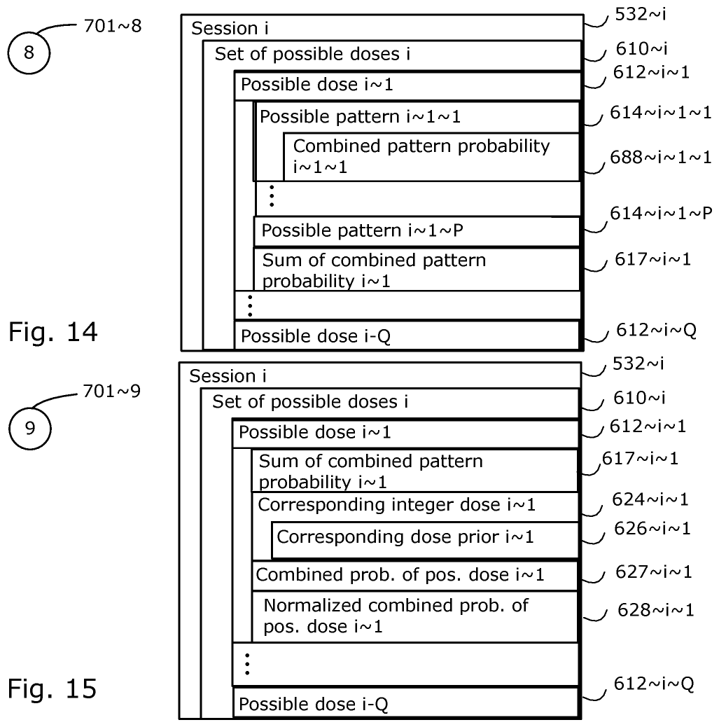

FIG. 14 shows a data structure illustrating the structuring a set of possible doses 610 comprising a number of possible doses 612. Each possible dose comprises one or more corresponding possible patterns 614, and each pattern comprises a combined pattern probability 688. If a possible dose comprises more than 1 possible pattern the possibility of each pattern is summed to provide a sum of combined pattern probability 617 for the possible dose in question. The sum of pattern probability 617 is calculated for each possible dose.

Update Dose Probabilities

Block 726. The dose probabilities obtained in step 724 is multiplied with the prior distribution from step 714 and renormalized so it sums to 1, which is known as Bayes' role. The resulting distribution is referred to as the “posterior” distribution over session dose.

The most likely session dose is the dose with the highest probability, i.e., argmax(posterior distribution). The argument which produces the maximum value. This is the best guess although it may or may not be a “good” guess. Determining the appropriateness of the estimate is the goal of the confidence score and evaluation described in step 728. The most likely dose in the posterior distribution is the Maximum Likelihood estimate.

FIG. 15 shows a data structure illustrating the structuring the possible doses with the corresponding sum of pattern probability 617 and the corresponding integer dose 624 and dose prior 626. The two probabilities both relate to the session dose size and can therefore be combined to a combined probability of possible doses 627. Again, this doses 627 can be normalized to sum to 1.

Calculate Confidence Score

Block 728. Intuitively, there are several ways to gauge confidence in the Maximum Likelihood of the estimate of the session dose:
(i) data probability. How large (or small) were the probabilities in step 720 before normalization?
(ii) Expected dose match. If there was an expected dose for this session, how far off is it from the Maximum Likelihood dose estimate?
(iii) Ambiguity. Did the Maximum Likelihood dose estimate dominate in the posterior session-dose distribution, or were there other peaks not far below it?
(iv) Priming consistency. Tracing the Maximum Likelihood dose estimate back to the winning pattern, e.g. “pi” or “ii”, does this match the user's historical priming propensity?

These four exemplary confidence metrics (more may be added) can be converted into confidence scores and are individually configurable using the ConfidenceWeight parameters. The overall confidence score is min(conf. score 1, conf. score 2, . . . )

The various confidence metrics are selected and the scores are tuned by setting the corresponding ConfidenceWeight variable. By convention, a confidence metric should have no effect when its corresponding weight is zero.

The overall confidence is min(all confidence metrics). The overall confidence is compared against a threshold (confidenceThreshold) to determine whether to label the session-injected dose or throw it back to the user interphase for the user to label manually.

Use the probability of the observed session data, evaluated at the Metric, maximum-likelihood dose. The confidence score formula is:

pDataConfidence=1−(pDataConfidenceWeight*(1−pData)),

so pDataConfidence=pData when the weighting is 1.0. pDataConfidenceWeight=1.0,

Metric, the difference between the expected dose and the maximum-likelihood dose as a confidence metric? The confidence score follows the formula:

expDoseConfidence=1−expDoseConfidenceWeight*(MLDose˜ExpDose)/sqrt(Variance),

expDoseConfidenceWeight=0.15. Up to 2 std deviations, if conf threshold is 0.7.

Metric, ambiguity in the posterior session-injected dose probability output. The confidence score formula is:

ambigConfidence=max(0,1−(ambigConfidenceWeight*(1−max(pDoseOut))/max(pDoseOut))),

such that with a weight of 1, ambigConfidence drops to zero when the maximum-likelihood dose probability falls to 0.5 (implying the sum of the other dose probabilities is also 0.5). ambigConfidenceWeight=0.75.

Metric, priming/non-priming consistency as a confidence metric? The score determines at what value of primeProb or 1-primeProb the confidence starts to decrease toward zero. E.g. 0 means consistencyConfidence will always be 1, 1 means consistencyConfidence=1 only when their priming or nonpriming history is perfect, and 0.5 means consistencyConfidence=1, as long as their priming history is somewhere between 50/50 and perfect. consistencyConfidenceWeight=0.5.

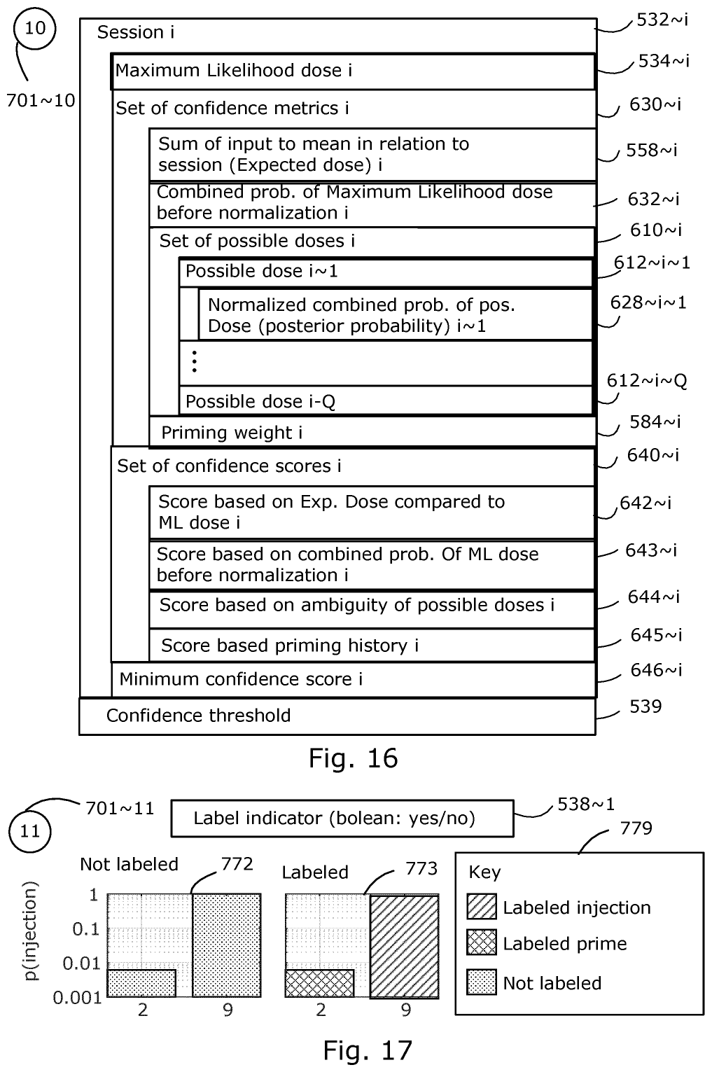

FIG. 16 shows a data structure for structuring the confidence metrics comprising a set of confidence metrics 630, and the corresponding confidence scores in a set of confidence scores 640. All the confidence scores 642, 643, 644, 645 are evaluated, and the minimum confidence score is evaluated against a confidence threshold 539.

Label Session if Confidence>Confidence Threshold

Block 730. The minimum confidence score is compared against a confidence threshold 539, which for example can be 0.7. If the minimum confidence score is greater than the confidence threshold, the session is “labelled” and the user does not need to be asked for confirmation. If the confidence score is less than the confidence threshold, the user interphase will need to ask the user to confirm the injected dose. Intelligent feedback can be adapted to, depending on the reason for the confidence being low, e.g., ask if it is priming consistency, the user could be asked “Did you forget to prime?” etc. It is important to remember that the labelling rate on a given dataset is not an intrinsic property of the algorithm. Labelling rate can easily be chosen anywhere from 0-100% depending on the confidence threshold value. It is advisable to choose a target labelling rate based on a trade-off between dose-estimation accuracy and user acceptance.

FIG. 17 illustrates schematically an unlabelled session 772 in a session detail plot, where the pattern of two dispenses is unknown. The figure also illustrates a labelled session 773, where the dispenses have been labelled with the most likely pattern providing the Maximum-Likelihood dose. A label indicator 538 can indicate whether or not to label a session depending on the outcome of the confidence score evaluation.

### Example 3

Sectioning of Time

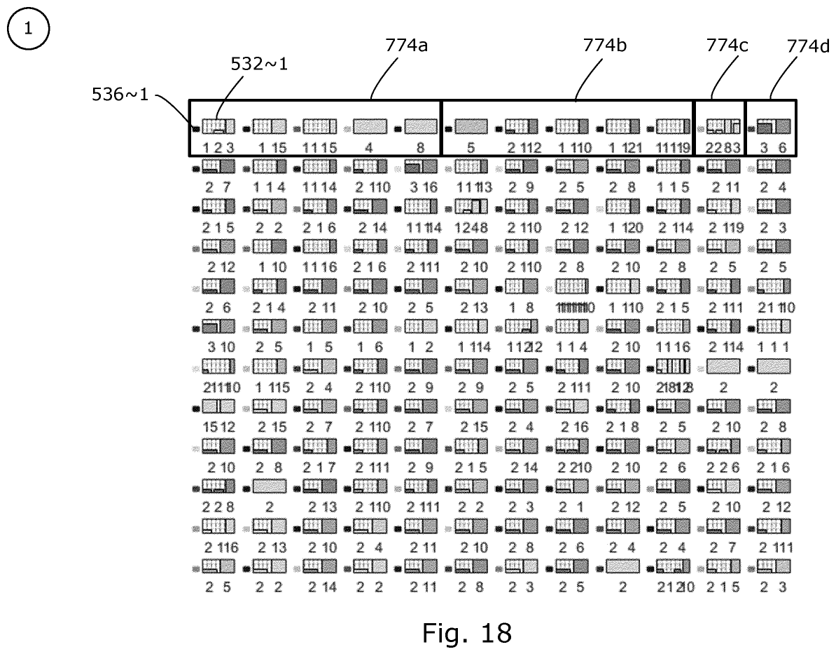

FIG. 18 shows a patient survey plot. The survey plot shows the first up to 144 sessions with 144 session detail plots. The first 5 sessions 774a are not labelled, the following 5 sessions 774b are labelled, the session 774c is not labelled and 774d is labelled. The rectangles between the session detail blots indicate the time between sessions. Due to the grey scaling it is not possible to identify the colour indicates, that would otherwise indicate “labelled”, “not labelled”, “injection”, “prime”. The numbers below the session detail blot indicate the size of the dispensed medicament. FIG. 18 relates to step 710 of sectioning dispenses into sections.

Determine Expected Dose

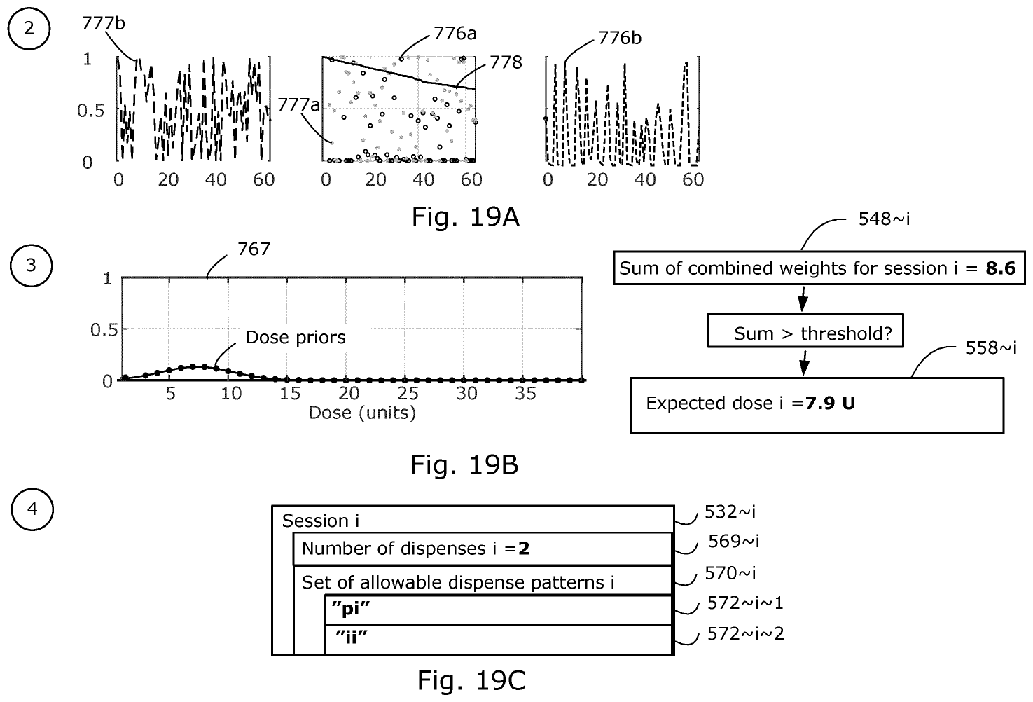

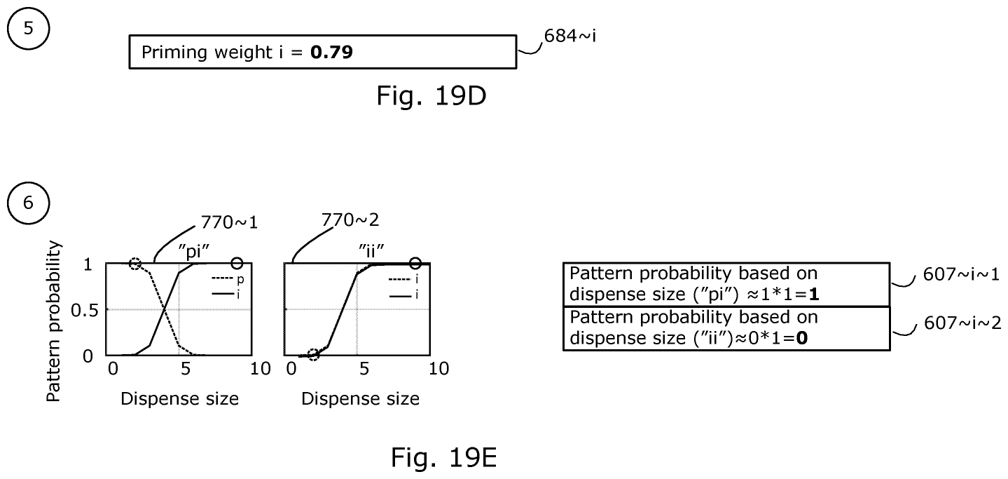

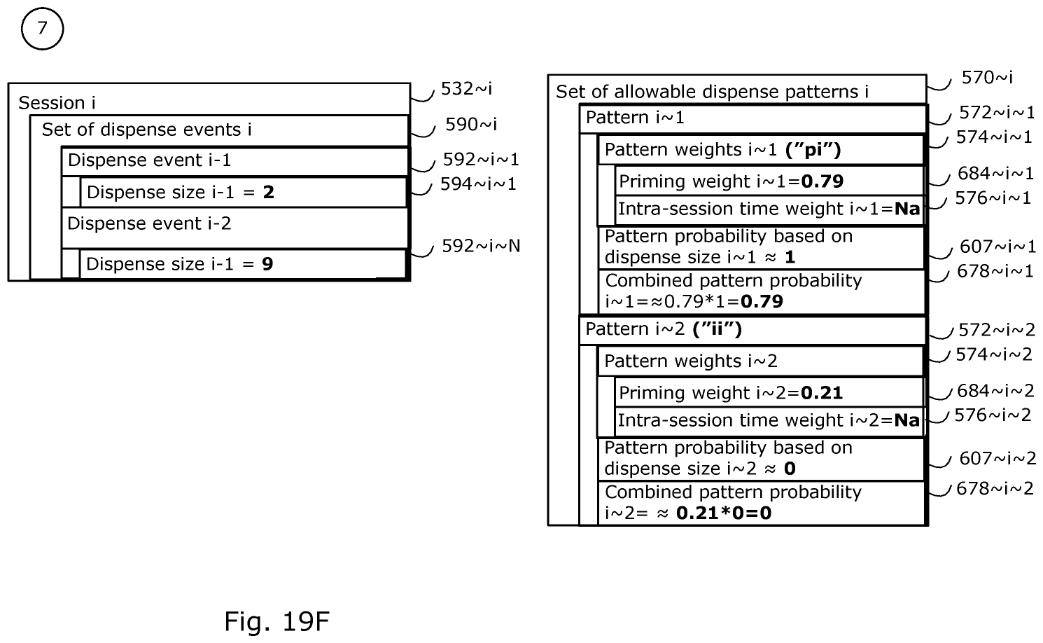

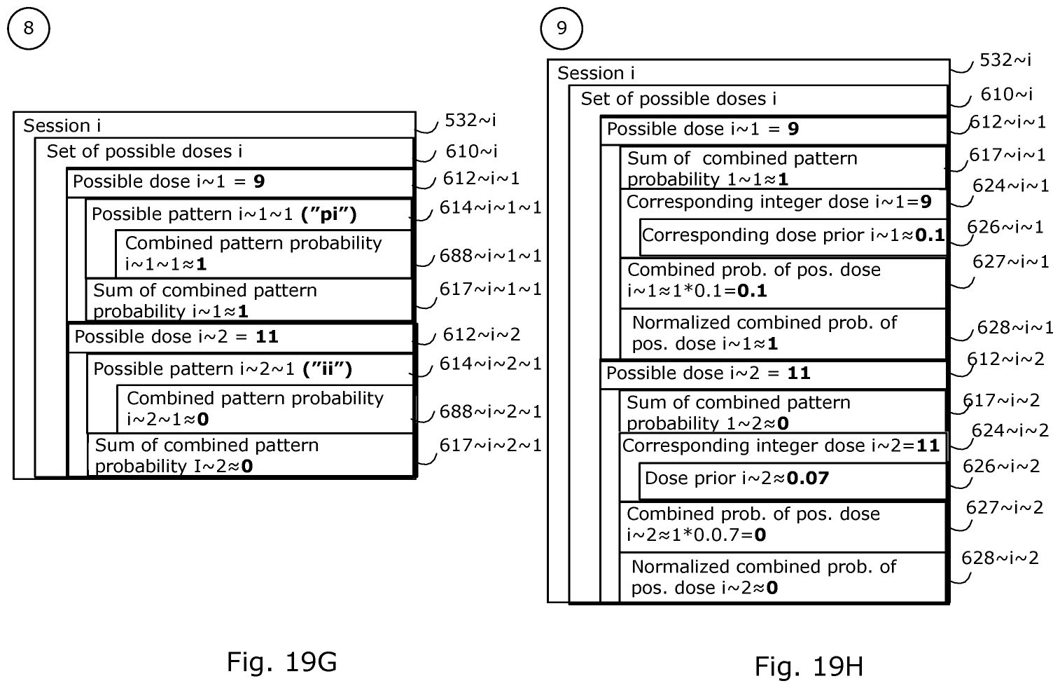

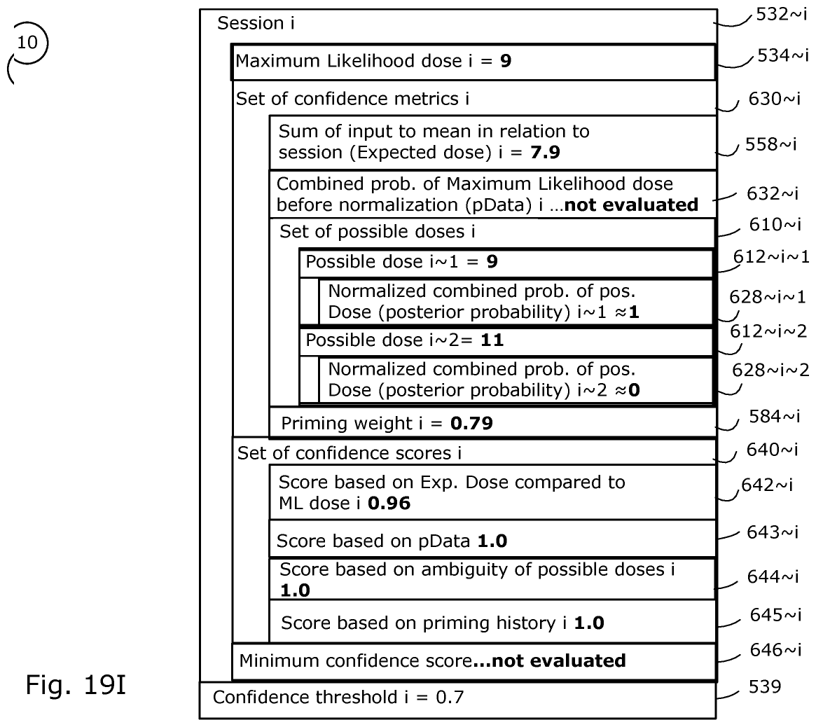

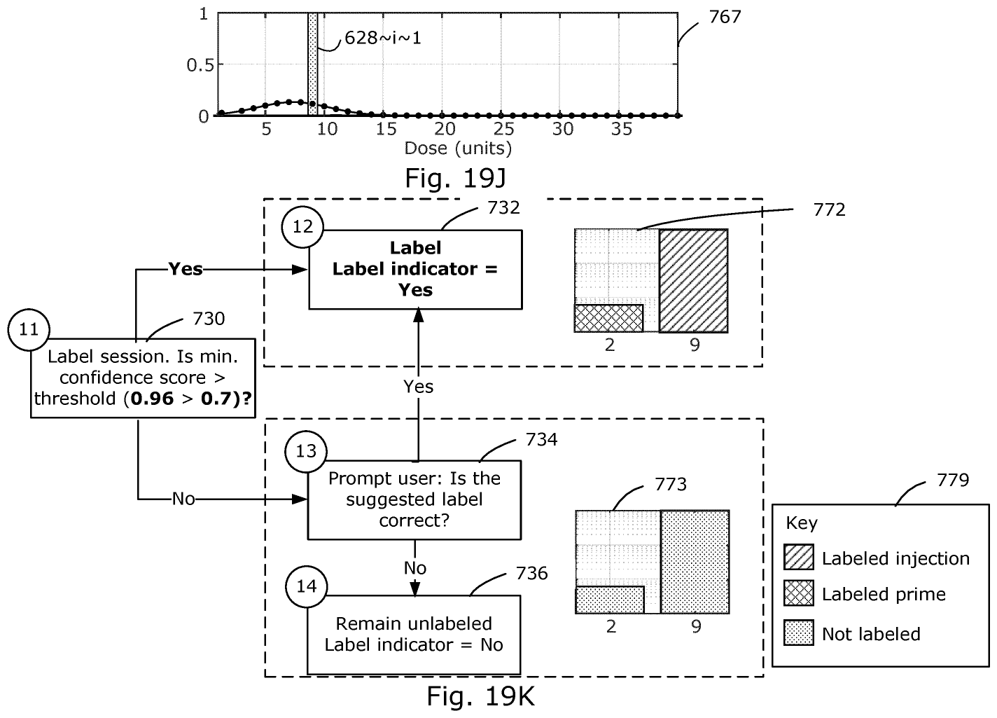

FIG. 19A relates to step 712 and shows a priori information in a “history weights” plot for the 144 sessions shown in the survey plot. The history weights plot summarizes the applicability of past sessions, sorted from newest to oldest, to the current session. The solid line 778 shown in the middle panel, is the weight due to session age, which, because of the sorting, is always a decreasing curve. The long dashed line 777b in the left panel and the corresponding solid dots 777a in the middle panel, is the weight due to similarity in inter-session gap length. The short dashed line 776b in the right panel and the corresponding solid dots 776a in the middle panel, is the weight due to similarity in time of day. The more points close to 1.0, the more applicable those past sessions are when estimating the “expected dose”.

The sum of all the combined weights 548 (here: 8.6) in relation to the current session, must exceed a threshold before an expected dose can be computed. The sum of the weights 548 are shown in a data structure in FIG. 19B left panel.

Set Dose Probabilities

In this example, there is sufficient history (8.6 is above the empiric threshold) to compute the weighted average, which is the “expected dose” 558. The expected dose is in this case 7.9 U. The prior dose probabilities are Gaussian, shown with dots and a solid line in the plot 767 in FIG. 19B. The mean is at the expected dose 558 and the variance is proportional to the variance of the past doses in the weighted average, the calculation of the prior distribution relates to step 714.

Allowed Patterns

FIG. 19C shows a data structure with the number of dispenses 569, and the set allowable patterns 570 comprising the allowable patterns 570.

Set Pattern Weights

FIG. 19D show a data structure with the pattern weights. Only the pattern weights relating to priming can be used, as there are only two dispenses in the session. Each pattern gets a weight based on the past priming behaviour. In this case, the patient is a very consistent primer, so we are strongly biased towards “pi” before we even consider the dispense sizes. FIG. 19D shows that the priming weight 684 in this case is 0.79, if we consider the weight as a fraction, however, a weight considering exponential decay with the fraction as argument is also possible depending on how easy or difficult is should be to change priming habit.

Calculate Pattern Probabilities

The dispenses in the session have the dispense sizes {2, 9}. By applying the discussed rules there are only two possible interpretations of the pattern 572, “pi” and “ii”. Each patter is illustrated with probability plot in FIG. 19E. The probability of the pattern “pi” is approximately 1, and the probability of the pattern ii is approximately 0. This is illustrated as pattern probabilities 607 in a data structure in FIG. 19E.

Pattern probabilities can be evaluated based on dispense size. Separate from the pattern weights, each pattern gets a probability based on the dispense sizes. Larger dispenses are more likely to be injections. The probability plots in FIG. 19E show P(prime) or P(injection) vs dose size for each dispense. For bolus drugs, the curves are fixed and cross 50/50 probability at 4 u. The bubbles are the points where the curves are evaluated, i.e. 2 u and 9 u. P(2 is a prime)*P(9 is an injection) is close to 1, so “pi” gets a high probability. P(2 is an injection) is very small, so ii has low probability.

Update Pattern Probabilities

FIG. 19F illustrates the step 722 of updating the pattern probabilities. In the left panel is shown the set of dispense events 590 for the current session i, with the corresponding dispense sizes 592. In the right panel is shown the set of allowable dispense patterns 570 for the current session i. The pattern probabilities based on dispense size are updated with the pattern weights to arrive at the overall pattern probabilities, essentially 100% for “pi” and 0% for “ii”. Weights based on intra-session time are not available for sessions with only two dispenses.

Convert Pattern Probabilities to Dose Probabilities

FIG. 19G shows a data structure comprising the set of possible doses 610, wherein each possible dose 612 comprises a possible pattern. FIG. 19G is related to the step 724 of converting pattern probabilities into dose probabilities. If P(“pi”)=x and P(“ii”)=y, then P(dose is 9)=x, P(dose is 11)=y, and P(dose is neither 9 nor 11)=0.

Update Dose Probabilities

Next, we update these dose probabilities by multiplying with the dose prior and renormalizing (Bayes' rule). The result is the final (“posterior”) dose probability distribution 617. The largest posterior dose probably is called the “maximum likelihood” dose.

FIG. 19H show a data structure comprising the set of possible doses 610 for the current session 532˜i, wherein each possible dose 612 comprises the sum of combined pattern probability 617, a corresponding integer dose 624 and dose prior 626 obtained in step 714. The probabilities 617, 626 are then combined to a combined probability of the possible dose 627. The combined probabilities of the possible dose 627 are normalized to sum to 1 to obtain the normalized combined probability of the possible dose. It is noticed that the Normalized combined probability of the possible dose 628 for the possible dose 612 being 9 is 1, i.e., the Maximum Likelihood dose is 9 U.

Calculate Confidence Scores

Confidence scores. We now have a dose estimate (9 U), but how likely is it to be correct? This is the domain of confidence scores. There are currently four types of confidence scores, all tuneable: How closely does the estimate match the expected dose? How likely are the selected pattern and dose probabilities before normalization (the pData number)? How ambiguous is the final (posterior) probability? i.e. is there >1 answer with nontrivial probability? Is the selected pattern consistent with this patient's priming history? The overall confidence is the smallest of these scores, in this case “ExpDose” (expected dose match). The overall confidence level of 0.96 is larger than the threshold 0.7, and the session is labelled and the session-dose is assigned the value of 9 U. Even though a session is not labelled it can still be used in the evaluation of later dose estimates in later sessions.

### SUMMARY

The session {2, 9} can only be interpreted in two ways. Because 2 is small for an injection but typical of a prime, and because this patient has been priming consistently, “pi” is much more likely than ii. The estimated dose is quite close to the dose average for this time of day and inter-session gap, the probabilities are high, there is no ambiguity in the final probabilities, and the priming behaviour is consistent, therefore, confidence is high. Because confidence is above our threshold (70% in the simulation), the session is formally labelled.

1. A computing system for enhancing data quality of a query drug dispense data set, wherein the system comprises one or more processors and a memory, the memory comprising:
instructions that, when executed by the one or more processors, perform a method responsive to receiving a query request for enhancing dispense data quality, the instructions comprising the steps of:
a) obtaining a query dispense data set comprising a plurality of dispense records created over a time course, each respective dispense record representing a dispense event comprising:
(i) a dispense amount of a medicament, wherein the dispense amount is one of a priming amount [p] or an injection amount [i], each amount corresponding to a size,
(ii) a corresponding dispense timestamp,
b) segmenting the query dispense data set into one or more current sessions, each current session comprising a sequence of dispense events clustered in time according to a set of clustering criterions,
for each current session:
c) creating a list of possible dispense patterns in accordance with a set of pattern rules, wherein a dispense pattern is a sequence of dispense amounts, either priming or injection amounts,
d) for each pattern calculating a combined pattern weight being the product of weight factors for each dispense in the pattern, wherein each weight factor is determined in accordance with a weight factor vs dispense size function for the given dispense type and dispense size, wherein the larger the dispense size, the more likely it is to represent an injection event and the less likely it is to represent a priming event,
e) identifying a winning pattern as the pattern being the pattern having the highest combined pattern weight, and
f) storing in memory the corresponding dispense events labelled as either a priming or an injection event corresponding to the winning pattern.

2. The computing system as in claim 1, the instructions comprising the further step of:
obtaining a history dispense data set comprising a plurality of prior dispense records created over a prior time course,

3. The computing system as in claim 2, the instructions comprising the further steps for each current session:
generating mean and variance values for an expected total injected amount distribution based on history dispense data,
comparing the highest and the second-highest combined pattern weights, and if the pattern weights are within a given proximity of each other then identify an updated winning pattern as the pattern having the highest probability according to the generated distribution.

4. The computing system as in claim 3, the instructions comprising the further step for each current session:
calculating a history weight for the history dispense data upon which the expected total injected amount values are based, the history weight being based on relevance criteria, comprising one or more of:
age of data,
time-of-day similarity, and
inter-session gap similarity,
wherein no expected total injected amount values are generated unless the history weight reaches a given minimum threshold.

5. The computing system as in claim 1, the instructions comprising the further step for each current session:
determining a combined confidence value based on one or more confidence metrics from the group of confidence values comprising:
data confidence value based on the value of the highest combined pattern weight,
expected-amount confidence value based on the difference between estimated total injected amount and an expected total injected amount, if calculated,
ambiguity confidence value based on the probability proximity of the highest and the second-highest combined pattern weights according to the generated distribution, if generated, and
priming confidence value based on the consistency between priming behavior of the winning pattern and,
wherein, when the combined confidence value is above a given threshold value, then:
calculate an estimated total injected amount as the sum of all injection amounts in the winning pattern.

6. The computing system as in claim 2, wherein, when the combined confidence value is above a given threshold value, then:
label the session corresponding to the winning pattern, wherein the mean and variance values for the expected total injected amount distribution is based on history dispense data from labeled sessions only.

7. The computing system as in claim 2, wherein a combined pattern weight is the product of one or more further factors, comprising:
priming probability factor based on history dispense data,
priming disparity factor, and
intra-session dispense interval factor for sessions having more than two dispenses.

8. The computing system as in claim 1, the instructions comprising the further step for each current session:
calculate an estimated total injected amount as the sum of all injection amounts in the winning pattern.

9. The computing system as in claim 1, wherein:
the obtained dispense records comprise an identifier for identifying a given dispense event as a bolus event or a basal event, and
the rules and parameters of the method is adapted for use with dispense data generated in a bolus only, basal only, or bolus and basal regimen.

10. The computing system as in claim 1, wherein:
the segmenting is controlled by a set of time parameters and a set of time measures, wherein the initial dispense event in the sequence of dispense events starts a session and zeros a timer, and the next dispenses are automatically included in this session until a session time window have elapsed, and wherein later dispenses are included, provided that the expressions: (i) the ratio between a resulting session length and the resulting inter-session length on either side of the session is less than the session length ratio, and (ii) the resulting session length is less than session window max, is true,
wherein the sequence of dispense events in the session defines a set of dispense events, and wherein each dispense event comprises a corresponding dispense size being the amount of dispensed medicament, and
wherein a new session is started, in response to the expressions are no longer true.

11. The computing system as in claim 1, wherein a given event or session label can be changed by a user.
